SEQUENCE LISTING (20 sequences)

SEQ ID NO: 1 - protein, 1130 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRCPPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTVININSSDEEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPTEPDS

SEQ ID NO: 2 - DNA, 3393 bp, Homosapiens
atggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgttccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagtttggagctcccgtgtccgtccctgtgcaggaaatcctggatttcatctgccggaccctcagcgtcagtagcaagaatattagcttgcatggagatggtcccctgcggctgctgctgctgccctctatccaccttgaggccttggacctgctgtctgcactcatcctcgcgtgtggaagccggctcttgcgctttgggatcctgatcggccgcctgcttccccaggtcctcaattcctggagcatcggtagagattccctctctccaggccaggagaggccttacagcacggttcggaccaaggtgtatgcgatattagagctgtgggtgcaggtttgtggggcctcggcgggaatgcttcagggaggagcctctggagaggccctgctcacccacctgctcagcgacatctccccgccagctgatgcccttaagctgcgtagcccgcgggggagccctgatgggagtttgcagactgggaagcctagcgcccccaagaagctaaagctggatgtgggggaagctatggccccgccaagccaccggaaaggggatagcaatgccaacagcgacgtgtgtgcggctgcactcagaggcctcagccggaccatcctcatgtgtgggcctctcatcaaggaggagactcacaggagactgcatgacctggtcctccccctggtcatgggtgtacagcagggtgaggtcctaggcagctccccgtacacgagctcccgctgccgccgtgaactctactgcctgctgctggcgctgctgctggccccgtctcctcgctgcccacctcctcttgcctgtgccctgcaagccttctccctcggccagcgagaagatagccttgaggtctcctctttctgctcagaagcactggtgacctgtgctgctctgacccacccccgggttcctcccctgcagcccatgggccccacctgccccacacctgctccagttccccctcctgaggccccatcgcccttcagggccccaccgttccatcctccgggccccatgccctcagtgggctccatgccctcagcaggccccatgccctcagcaggccccatgccctcagcaggccctgtgccctcggcacgccctggacctcccaccacagccaaccacctaggcctttctgtcccaggcctagtgtctgtccctccccggcttcttcctggccctgagaaccaccgggcaggctcaaatgaggaccccatccttgcccctagtgggactcccccacctactatacccccagatgaaacttttggggggagagtgcccagaccagcctttgtccactatgacaaggaggaggcatctgatgtggagatctccttggaaagtgactctgatgacagcgtggtgatcgtgcccgaggggcttccccccctgccacccccaccaccctcaggtgccacaccaccccctatagcccccactgggccaccaacagcctcccctcctgtgccagcgaaggaggagcctgaagaacttcctgcagccccagggcctctcccgccacccccacctccgccgccgcctgttcctggtcctgtgacgctccctccaccccagttggtccctgaagggactcctggtgggggaggacccccagccctggaagaggatttgacagttattaatatcaacagcagtgatgaagaggaggaggaagaggaagaaggggaagaagaagaagaggaagaagaggaagaggaggaagactttgaggaagaggaagaggatgaagaggaatattttgaagaggaagaagaggaggaagaagagtttgaggaagaatttgaggaagaagaaggtgagttagaggaagaagaagaagaggaggatgaggaggaggaagaagaactggaagaggtggaagacctggagtttggcacagcaggaggggaggtagaagaaggtgcacctccacccccaaccctgcctccagctctgcctccccctgagtctcccccaaaggtgcagccagaacccgaacccgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagcctgaggatgggagtgacaaggtgcagcccccaccagagacacctgcagaagaagagatggagacagagacagaggccgaagctctccaggaaaaggagcaggatgacacagctgccatgctggccgacttcatcgattgtccccctgatgatgagaagccaccacctcccacagagcctgactcctag

SEQ ID NO: 3 - protein, 253 aa, Homo sapiens
LTVININSSDEEEEEEEEGEEEEEEEEEEEEDFEEEEEDEEEYFEEEEEEEEEFEEEFEEEEGELEEEEEEEDEEEEEELEEVEDLEFGTAGGEVEEGAPPPPTLPPALPPPESPPKVQPEPEPEPGLLLEVEEPGTEEERGADTAPTLAPEALPSQGEVEREGESPAAGPPPQELVEEEPSAPPTLLEEEPEDGSDKVQPPPETPAEEEMETETEAEALQEKEQDDTAAMLADFIDCPPDDEKPPPPTEPDS

SEQ ID NO: 4 - DNA, 762 bp, Homo sapiens
ttgacagttattaatatcaacagcagtgatgaagaggaggaggaagaggaagaaggggaagaagaagaagaggaagaagaggaagaggaggaagactttgaggaagaggaagaggatgaagaggaatattttgaagaggaagaagaggaggaagaagagtttgaggaagaatttgaggaagaagaaggtgagttagaggaagaagaagaagaggaggatgaggaggaggaagaagaactggaagaggtggaagacctggagtttggcacagcaggaggggaggtagaagaaggtgcacctccacccccaaccctgcctccagctctgcctccccctgagtctcccccaaaggtgcagccagaacccgaacccgaacccgggctgcttttggaagtggaggagccagggacggaggaggagcgtggggctgacacagctcccaccctggcccctgaagcgctcccctcccagggagaggtggagagggaaggggaaagccctgcggcagggccccctccccaggagcttgttgaagaagagccctctgctcccccaaccctgttggaagaggagcctgaggatgggagtgacaaggtgcagcccccaccagagacacctgcagaagaagagatggagacagagacagaggccgaagctctccaggaaaaggagcaggatgacacagctgccatgctggccgacttcatcgattgtccccctgatgatgagaagccaccacctcccacagagcctgactcctag

SEQ ID NO: 5 - protein, 445 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGAS

SEQ ID NO: 6 - DNA, 990 bp, Homo sapiens
atggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgttccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagttt

SEQ ID NO: 7 - protein, 15 aa, Homo sapiens
SSGPRLRLLLLESVS

SEQ ID NO: 8 - protein, 15 aa, Homo sapiens
PHLPGLMCLLRLHGS

SEQ ID NO: 9 - protein, 15 aa, Homo sapiens
FEGLCLLSLLVGESP

SEQ ID NO: 10 - protein, 15 aa, Homo sapiens
LAVAVLRDLLRYAAQ

SEQ ID NO: 11 - protein, 15 aa, Homo sapiens
ISMNHLPGLLTSLLG

SEQ ID NO: 12 - protein, 22 aa, Homo sapiens
SWEQELHSLLASLHTLLGALYE

SEQ ID NO: 13 - protein, 17 aa, Homo sapiens
HGDGPLRLLLLPSIHLE

SEQ ID NO: 14 - protein, 880 aa, Homo sapiens
MAAAVLSGPSAGSAAGVPGGTGGLSAVSSGPRLRLLLLESVSGLLQPRTGSAVAPVHPPNRSAPHLPGLMCLLRLHGSVGGAQNLSALGALVSLSNARLSSIKTRFEGLCLLSLLVGESPTELFQQHCVSWLRSIQQVLQTQDPPATMELAVAVLRDLLRYAAQLPALFRDISMNHLPGLLTSLLGLRPECEQSALEGMKACMTYFPRACGSLKGKLASFFLSRVDALSPQLQQLACECYSRLPSLGAGFSQGLKHTESWEQELHSLLASLHTLLGALYEGAETAPVQNEGPGVEMLLSSEDGDAHVLLQLRQRFSGLARCLGLMLSSEFGAPVSVPVQEILDFICRTLSVSSKNISLHGDGPLRLLLLPSIHLEALDLLSALILACGSRLLRFGILIGRLLPQVLNSWSIGRDSLSPGQERPYSTVRTKVYAILELWVQVCGASAGMLQGGASGEALLTHLLSDISPPADALKLRSPRGSPDGSLQTGKPSAPKKLKLDVGEAMAPPSHRKGDSNANSDVCAAALRGLSRTILMCGPLIKEETHRRLHDLVLPLVMGVQQGEVLGSSPYTSSRCRRELYCLLLALLLAPSPRCPPPLACALQAFSLGQREDSLEVSSFCSEALVTCAALTHPRVPPLQPMGPTCPTPAPVPPPEAPSPFRAPPFHPPGPMPSVGSMPSAGPMPSAGPMPSAGPVPSARPGPPTTANHLGLSVPGLVSVPPRLLPGPENHRAGSNEDPILAPSGTPPPTIPPDETFGGRVPRPAFVHYDKEEASDVEISLESDSDDSVVIVPEGLPPLPPPPPSGATPPPIAPTGPPTASPPVPAKEEPEELPAAPGPLPPPPPPPPPVPGPVTLPPPQLVPEGTPGGGGPPALEEDLTV

SEQ ID NO: 15 - DNA, 2636 bp, Homo sapiens
atggcggcagccgttctgagtgggccctctgcgggctccgcggctggggttcctggcgggaccgggggtctctcggcagtgagctcgggcccgcggctccgcctgctgctgctggagagtgtttctggtttgctgcaacctcgaacggggtctgccgttgctccggtgcatcccccaaaccgctcggccccacatttgcccgggctcatgtgcctattgcggctgcatgggtcggtgggcggggcccagaacctttcagctcttggggcattggtgagtctcagtaatgcacgtctcagttccatcaaaactcggtttgagggcctgtgtctgctgtccctgctggtaggggagagccccacagagctattccagcagcactgtgtgtcttggcttcggagcattcagcaggtgttacagacccaggacccgcctgccacaatggagctggccgtggctgtcctgagggacctcctccgatatgcagcccagctgcctgcactgttccgggacatctccatgaaccacctccctggccttctcacctccctgctgggcctcaggccagagtgtgagcagtcagcattggaaggaatgaaggcttgtatgacctatttccctcgggcttgtggttctctcaaaggcaagctggcctcattttttctgtctagggtggatgccttgagccctcagctccaacagttggcctgtgagtgttattcccggctgccctctttaggggctggcttttcccaaggcctgaagcacaccgagagctgggagcaggagctacacagtctgctggcctcactgcacaccctgctgggggccctgtacgagggagcagagactgctcctgtgcagaatgaaggccctggggtggagatgctgctgtcctcagaagatggtgatgcccatgtccttctccagcttcggcagaggttttcgggactggcccgctgcctagggctcatgctcagctctgagtttggagctcccgtgtccgtccctgtgcaggaaatcctggatttcatctgccggaccctcagcgtcagtagcaagaatattagcttgcatggagatggtcccctgcggctgctgctgctgccctctatccaccttgaggccttggacctgctgtctgcactcatcctcgcgtgtggaagccggctcttgcgctttgggatcctgatcggccgcctgcttccccaggtcctcaattcctggagcatcggtagagattccctctctccaggccaggagaggccttacagcacggttcggaccaaggtgtatgcgatattagagctgtgggtgcaggtttgtggggcctcggcgggaatgcttcagggaggagcctctggagaggccctgctcacccacctgctcagcgacatctccccgccagctgatgcccttaagctgcgtagcccgcgggggagccctgatgggagtttgcagactgggaagcctagcgcccccaagaagctaaagctggatgtgggggaagctatggccccgccaagccaccggaaaggggatagcaatgccaacagcgacgtgtgtgcggctgcactcagaggcctcagccggaccatcctcatgtgtgggcctctcatcaaggaggagactcacaggagactgcatgacctggtcctccccctggtcatgggtgtacagcagggtgaggtcctaggcagctccccgtacacgagctcccgctgccgccgtgaactctactgcctgctgctggcgctgctgctggccccgtctcctcgctgcccacctcctcttgcctgtgccctgcaagccttctccctcggccagcgagaagatagccttgaggtctcctctttctgctcagaagcactggtgacctgtgctgctctgacccacccccgggttcctcccctgcagcccatgggccccacctgccccacacctgctccagttccccctcctgaggccccatcgcccttcagggccccaccgttccatcctccgggccccatgccctcagtgggctccatgccctcagcaggccccatgccctcagcaggccccatgccctcagcaggccctgtgccctcggcacgccctggacctcccaccacagccaaccacctaggcctttctgtcccaggcctagtgtctgtccctccccggcttcttcctggccctgagaaccaccgggcaggctcaaatgaggaccccatccttgcccctagtgggactcccccacctactatacccccagatgaaacttttggggggagagtgcccagaccagcctttgtccactatgacaaggaggaggcatctgatgtggagatctccttggaaagtgactctgatgacagcgtggtgatcgtgcccgaggggcttccccccctgccacccccaccaccctcaggtgccacaccaccccctatagcccccactgggccaccaacagcctcccctcctgtgccagcgaaggaggagcctgaagaacttcctgcagccccagggcctctcccgccacccccacctccgccgccgcctgttcctggtcctgtgacgctccctccaccccagttggtccctgaagggactcctggtgggggaggacccccagccctggaagaggatttgac

SEQ ID NO: 16 - RNA, 42 nt, Artificial Sequence
  Artificially generated siRNA.
rggaggagccugaagaacuudraaguucuucaggcuccuccd

SEQ ID NO: 17 - RNA, 42 nt, Artificial Sequence
  Artificially generated siRNA.
ruuccuggagcaucgguagadrucuaccgaugcuccaggaad

SEQ ID NO: 18 - RNA, 42 nt, Artificial Sequence
  Artificially generated siRNA.
rggcaagcuggccucauuuudraaaaugaggccagcuugccd

SEQ ID NO: 19 - RNA, 42 nt, Artificial Sequence
  Artificially generated siRNA.
rggaaugaaggcuuguaugadrucauacaagccuucauuccd

SEQ ID NO: 20 - protein, 19 aa, Homo sapiens
RDSLSPGQERPYSTVRTKV

(19) United States
(12) Patent Application Publication
(10) Pub. No.: US 2005/0095241 A1
(43) Pub. Date: May 5, 2005

(54) Estrogen receptor modulators and uses thereof

(76) Inventors: Ratna K. Vadlamudi, Metairie, LA (US); Rakesh Kumar, Houston, TX (US); Seetharaman Balasenthil, Houston, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 10/927,644

(22) Filed: Aug. 26, 2004

Related U.S. Application Data
(60) Provisional application No. 60/498,118, filed on Aug. 26, 2003.

(51) Int. Cl. A61K 48/00; C07K 16/28; C07K 14/72; A61K 39/395

(52) U.S. Cl. 424/143.1; 514/12; 514/44; 530/350; 530/324; 530/388.22; 536/23.1

(57) ABSTRACT

The present disclosure relates to the field of cellular tumorigenesis and cancer biology. More specifically, the present disclosure relates to tumorigenesis and cancer in estrogen-responsive cell types, including cell types such as testis, ovary and uterine tissues, mammary gland, brain, skeletal muscle, and lung tissues. The present disclosure further relates to compositions including polypeptides, oligopeptides, petidomimetics, antibodies, and nucleic acids, and pharmaceutical compositions, diagnostic kits, and therapeutic kits useful in the diagnosis or treatment of tumorigenesis in estrogen-responsive cell types.

### CROSS-REFERENCE TO RELATED APPLICATIONS

This application claims benefit of priority to provisional application 60/498,118 filed Aug. 26, 2003.

### STATEMENT REGARDING FEDERALLY SPONSORED RESEARCH OF DEVELOPMENT

This invention was supported, in whole or in part, by National Institutes of Health Research Grant Nos. 9-7150741 and RO1 CA095681. The United States Government has certain rights in the invention.

### FIELD OF THE INVENTION

The present invention relates to the field of pharmaceuticals and tumor therapies. More particularly, the invention relates to estrogen receptor modulation and pharmaceutical compositions effective in treating hormone-dependent tumors.

### BACKGROUND OF THE INVENTION

Nuclear hormone receptors (NRs) constitute a large family of transcription factors that regulate gene expression in a ligand-dependent manner. NRs play an important role in vertebrate development and have been implicated in a broad range of cellular responses such as differentiation, proliferation, and homeostasis. Kliewer, S. A. et al., Science 284, 757-760 (1999); Xu, L. et al., Curr. Opin. Genet. Dev. 9, 140-147 (1999). Currently, the NR superfamily is divided into three subfamilies. Type I includes steroid hormone receptors, such as estrogen, progestin, androgen or glucocorticoid receptors. Type II includes non-steroidal hormone receptors, such as retinoic acid, thyroid hormone, and vitamin D receptors. Type III currently includes orphan receptors that do not have a well characterized ligand. McKenna, N. J. et al., Endocr. Rev. 20, 321-344 (1999).

NRs share several structural features including an N-terminal ligand-independent transcriptional activation function domain 1 (Af1); a highly conserved central DNA binding domain (DBD) that targets the NR to specific DNA motifs; a C-terminal ligand binding domain (LBD); and a C-terminal ligand-dependent transcriptional activation finction domain (AF2). McKenna, N. J. (1999); Tsai, M. J. et al., Annu. Rev. Biochem. 63, 451-486 (1994). Hormone binding to a NR triggers a conformational change allowing the NR to bind responsive elements in the target gene promoters. LBDs of the NRs are diverse in sequence, accounting for ligand diversity, but share a similar overall three-dimensional structure. McKenna, N. J. (1999) AF2 is highly conserved among various NRs, but AF1 is not conserved. Xu, L. (1999).

Transcriptional activity of the NRs is affected by several regulatory coactivators and corepressors in addition to the hormones. McKenna, N. J. (1999); Glass, C. K, et al., Curr. Opin. Cell Biol. 9, 222-232 (1997). The coactivators do not normally bind to DNA, but are recruited to the target gene promoters through protein-protein interactions with the NRs. Ma, H. et al., Mol. Cell. Biol. 19, 6164-6173 (1999). The p160 family is a well-studied family of NR coactivators, including steroid receptor coactivator SRC1, glucocorticoid receptor-interacting protein GRIP1/FIF2, and P/CIP (also known as AIB1, TRAM1, or RAC3). Torchia, J. et al., Curr. Opin. Cell Biol. 10, 373-383 (1998). A second coactivator family includes the cAMP response element-binding protein CBP and p300. Chakravarti, D. et al., Nature 383, 99-103 (1996). Among the corepressors, nuclear receptor corepressor (NcoR) and silencing mediator for retinoic and thyroid receptor (SMRT) have been widely characterized and implicated in transcriptional silencing of genes that are normally responsive to receptors of thyroid hormone, retinoic acid, retinoid X, and vitamin D in the absence of ligand. McKenna, N. J. (1999) Corepressors have been shown to associate preferentially with antagonist-occupied NRs. Wagner, B. L. et al., Mol. Cell. Biol. 18, 1369-1378 (1998). A few bifunctional coregulators that can act as both coactivators and corepressors of NRs have also been reported including mouse zinc finger protein, a regulator of apoptosis and cell cycle arrest (ZAC1) (Huang, S. M. and Stallcup, M. R., Mol. Cell. Biol. 20, 1855-1867 (2000)); a NR-binding set domain-containing protein (NSD1) (Huang, N. et al., EMBO J. 17, 3398-3412 (1998)); and RIP140 (Cavailles, V. et al., EMBO J. 14, 8741-3751 (1995)).

Evidence suggests that multiprotein complexes containing coactivators, NRs and transcriptional regulators assemble in response to hormone binding and activate transcription. McKenna, N. J. (1999) Researchers are also actively investigating the molecular mechanisms whereby hormones elicit tissue type- and cell type-specific responses and the composition of coactivator proteins involved. Structural analysis of coactivators has identified a motif consisting of five amino acids LXXLL (where X is any amino acid) which is sufficient to mediate coregulator binding to ligand-NR complexes. Heery, D. M. et al., Nature 387, 733-736 (1997). Coactivators SRC1, CBP, and p300 have intrinsic histone acetyltransferase activity (Spencer, T. E. et al., Nature 389, 194-198 (1997)), while NcoR and SMRT associate with histone deacetylases and mSin3A. Nagy, L. et al., Cell 89, 373-380 (1997). Association of histone acetyltransferase and deacetylase activities with coregulators suggest that modulation of chromatin structures constitutes a potential mechanism of coregulator function. Xu, L. (1999). Another mechanism of gene transcription regulation involves the phosphorylation of the coregulators. Glass, C. K. and Rosenfeld, M. G., Genes Dev. 14, 121-141 (2000).

Steroid hormone 17β-estradiol (E₂) plays an important role in controlling the expression of genes involved in a wide variety of biological processes, including development, homeostasis, regulation of the cardiovascular system, the determination of bone density, and breast tumor progression. Couse, J. F. and Korach, K S., Endocr. Rev. 20, 358-417 (1999). The biological effects of estrogen are mediated by its binding to the structurally and finctionally distinct estrogen receptors, ERα and ERβ. ERα is the major ER in mammary epithelium. Warner, M. et al., Curr. Opin. Obstet. Gynecol. 11, 249-254 (1999). Like other steroid receptors, ERα comprises an N-terminal AF1, a DBD, and a C-terminal LBD containing an AF2 domain. Kumar, V. et al., Cell 51, 941-951 (1987). Upon binding of E2 to ERα, the ligand-activated ERα translocates to the nucleus, binds to the 13-base pair palindromic estrogen response enhancer element (ERE) of the target genes, and stimulates gene transcription, thus promoting the growth of breast cancer cells. Dubik, D. and Shiu, R. P., J. Biol. Chem. 263, 12705-12708 (1988). Several of the diverse functions of the estrogens depend on differential recruitment of coregulators to the E₂-ER complex. Transcription functions of the ER can be influenced by several coregulators, including SRC1, GRIP1, AIB1, CBP/p300, TIF1, PGC1, and DAX1. McKenna, N. J. (1999); Zhang, J. et al., J. Biol. Chem. 275, 39855-39859 (2000); Tcherepanova, I. et al., J. Biol. Chem. 275, 16302-16308 (2000). Although much is known about the structure of coregulators, very little is known about the physiological role of coregulator proteins in the development, hormone regulation, and progression of cancer.

Antiestrogens and selective estrogen receptor modulators have been shown to effectively inhibit the growth of hormone-dependent tumor cells due largely to their antagonistic or antiestrogenic properties. Many patients that respond to antiestrogenic therapy, however, eventually develop a resistance to the treatment, becoming hormone-independent. Mechanisms involved in the progression, and eventual resistance, from hormone-dependence to hormone-independence in breast cancer are believed to include expression of variant or mutant ERs, ligand independent activation of ER, adaptation of tumors to lower concentrations of estrogen, and pharmacological alterations of the ER coregulators.

Proline, glutamic acid and leucine rich protein 1 (PELP1) has recently been identified as a member of the family of coregulators of NRs (Vadlamudi, et al., J. Biol. Chem. 276(41): 38272-38279 (2001), incorporated herein by reference in its entirety). The PELP1 polypeptide, as the name suggests, is unusually rich in the amino acids proline, glutamic acid, and leucine. The N-terminal region of PELP1 has nine LXXLL motifs. Id. LXXLL motifs have been shown to mediate ligand-dependent binding of coactivators with a NR. PELP1 is further characterized by a centrally located consensus nuclear localization motif starting with amino acids 495-498 (Id., see also Chelsky, D. et al. (1989) Mol. Cell. Biol. 9, 2487-2492). Flanking the central nuclear localization motif are two cysteine rich regions which potentially form three zinc-fingers. Id. The C-terminal region of PELP1 contains two proline-rich C-terminal regions (31% proline, amino acids 751-870; 23% proline, amino acids 970-1130) constituting transcriptional activation domains. Id. A region rich in acidic amino acids is located between these two proline rich regions. Id. PELP1 also contains several consensus phosphorylation sites. Id. PELP1 is expressed differentially in various estrogen responsive tissues including testis, ovary and uterine tissues, mammary gland, brain, skeletal muscle, and lung tissues. Id. Further, increased expression of PELP1 occurs in ovarian and uterine tumors, in addition to breast cancer cells.

PELP1 actively participates in the ER pathway as a ligand-dependent coactivator. PELP1 interacts with and works as a coactivator of both ERα and ERβ isoforms. The ER pathway has been implicated in the progression of breast cancer tumorigenesis and coregulators of that pathway play a role in tumor progression. Expression of PELP1 augments the transcriptional activity of the ER-E₂ complex. PELP1 is also overexpressed in breast tumor cells suggesting that PELP1 impacts the nuclear signaling pathways, specifically the estrogen receptor pathway, to modulate the responses of estrogen and anti-estrogen in hormone-dependent cancers. Further, PELP1 recruitment to the ER response element for transcription regulation is hormone dependent, indicating that overexpression of PELP1 hypersensitizes cells to estrogen levels. This PELP1-induced hypersensitization to reduced hormone levels is one mechanism leading to hormone-resistance in cancer cells. Blocking this hypersensitization provides a strong mechanism to slow or halt tumorigenesis in hormone resistant cancers.

Additional studies have demonstrated PELP1 involvement in cell cycle progression. (Setharaman B., and Vadlamudi, R. K. (2003) J. Biol. Chem. 278:22118-22127). Specifically, PELP1 overexpression in breast cancer cells hypersensitizes those cells to estradiol signaling leading to enhanced progression of breast cancer cells to the S phase of the cell cycle. This increased progression of breast cancer cells through the cell cycle has been linked to PELP1-mediated hyperphosphorylation of the cell cycle switch protein, retinoblastoma (pRb). Retinoblastoma phosphorylation plays a key role in cell cycle progression and it is well known in the art that increased pRb phosphorylation leads to progression of cells from the G1 phase to the S phase. Further, interactions between PELP1 and pRb also increase the expression of the Cyclin D1, a protein known to be deregulated in a number of breast cancers. Blocking the increased progression of cancer cells through the cell cycle also provides a desirable target to slow or halt the progression of tumorigenesis.

Currently, various selective estrogen-receptor modulators (SERMs) are used in the treatment of breast cancer. One disadvantage of SERMs, however, is a partial agonistic action of these compounds in non-targeted tissues. For example, tamoxifen, the most commonly prescribed SERM, is very effective in the treatment of breast cancer but also provides a stimulus for endometrial tumors. There is a need for effective therapeutics capable of blocking estrogenic responses regardless of tissue type.

Additional studies have localized PELP1 interactions with ER to several ER-interacting sites in the N-terninal region of PELP1. Expression of PELP1 lacking an activation domain or disrupting PELP1-ER interactions using small interfering RNAs (siRNAs) blocks tamoxifen-mediated agonistic responses in endometrial cells. Disruption of PELP1-ER interactions, therefore, provides an additional target to slow or halt tumorigenesis while successfully reducing the partial agonistic action of traditional SERMs in non-targeted tissues.

The role of PELP1 in both the ER signaling pathway and cell cycle makes it a desirable target for such therapeutics. Modulating or blocking PELP1 activity provides a strong mechanism to interfere with the progression of tumorigenesis in a variety of estrogen-responsive tissues including mammary, testis, ovarian and uterine tissues. Additionally, interfering with PELP1 activity is likely to block estrogenic responses regardless of the tissue type, unlike conventional anti-estrogenic compounds. PELP1 activity can also be used as a marker of hormonal hypersensitivity in tumors as a diagnostic tool.

### SUMMARY OF THE INVENTION

The present disclosure arises from several surprising discoveries related to alteration or modulation of PELP1 in tumor cells. First, deletion of amino acids from the C-terminal region of PELP1 (SEQ ID NO:1, full length PELP1; SEQ ID NO:3, C-terminal region of PELP1; and SEQ ID NO:14, C-terminal deleted PELP1 mutant, PELP1-H1) blocks estrogenic responses in tumor cells resulting in reduced tumorigenesis and cell cycle progression. Second, PELP1 interactions with the estrogen receptor have been localized to several ER-interacting sites located in the N-terminal region of the PELP1 protein (SEQ ID NO:5, N-terminal region of PELP1). Disruption of interactions between PELP1 and ER blocks tamoxifen-mediated agonist response in endometrial cells. Disruption of PELP1 activity, therefore, provides a novel pathway for therapeutic activity to slow or halt progression of tumorigenesis and cancer in estrogen-responsive cell types, including cell types such as testis, ovary and uterine tissues, mammary gland, brain, skeletal muscle, and lung tissues, as well as reducing the partial agonistic action of SERM's in non-targeted tissues.

One embodiment of the present invention relates to compositions comprising one or more polypeptides, oligopeptides, or peptidomimetics capable of disrupting PELP1 activity including, but not limited to, SEQ ID NOS:7-13 (PELP1-ER-blocking peptides) and variants thereof. Polypeptides, oligopeptides, or peptidomimetics according to the present invention can be obtained by any means known in the art, including isolation from natural sources, recombinant production in prokaryotic or eukaryotic host cells, or chemical synthesis.

In certain embodiments polypeptides or oligopeptides are produced by culturing host cells under conditions promoting expression and recovering the poly- or oligopeptide from the culture medium. Expression of these poly- or oligopeptides in prokaryotic or eukaryotic cells such as bacteria, yeast, plant, insect and animals cells is encompassed by the invention. In other embodiments, polypeptides, oligopeptides, or peptidomimetics are produced by chemical synthesis using methods well known in the art.

Another embodiment of the present invention provides small interfering RNAs (siRNAs), or antisense nucleic acids that modulate or disrupt PELP1 transcription or translation. siRNAs are typically less than 100 bp in length and preferably 30 bp or shorter. Examples of siRNAs according to the invention are provided at any of SEQ ID NOS:16-19. Antisense nucleic acids and siRNAs can be made by any approach known in the art including the use of complementary DNA strands or through chemical synthesis.

An alternative embodiment of the present invention is an isolated nucleic acid molecule encoding any of the polypeptides disclosed herein. The isolated nucleic acid sequences can be naturally occurring nucleic acid sequences derived from the PELP1 gene (SEQ ID NO:2, full length PELP1; or SEQ ID NO:15, PELP1 H1 mutant), the C-terminal region of the PELP1 gene (SEQ ID NO:4), the N-terminal region of the PELP1 gene (SEQ ID NO:6) or variations of such sequences that encode the disclosed polypeptides, including nucleic acids complementary to these sequences. Both single and double stranded DNA and RNA molecules are encompassed by the invention, as well as nucleic acid molecules that hybridize to a denatured, double-stranded DNA molecule of the invention. Also included are isolated nucleic acid molecules derived by mutagenesis of nucleic acid molecules comprising the sequence of any of SEQ ID NOS:2 (full length PELP1 gene), 4 (C-terminal region), 6 (N-terminal region), or 15 (PELP1 H1 mutant), allelic variants of any of SEQ ID NOS:2, 4, 6, or 15, and degenerate variants of any of SEQ ID NOS:2, 4, 6, or 15. Nucleic acids of the invention can also be chemically synthesized based on the desired amino acid code to be expressed.

Isolated nucleic acid molecules according to the invention are preferably contained in vectors, including expression vectors capable of directing expression of the polypeptide in an appropriate host cell. An appropriate host cell is one in which expression of the polypeptide from the expression vector results in a biologically finctional polypeptide. Biological function may be related to proper folding and structural stability of the polypeptide such that eukaryotic, and particularly mammalian cell expression is preferred. Isolated nucleic acids of the present invention can also be included in cell-specific gene therapy compositions in which the vector or delivery system used to deliver the gene is targeted to a particular cell type and results in stable expression of the polypeptide in the targeted cell.

In certain embodiments, the present invention provides antibodies that bind with high specificity to the PELP1 polypeptide. Antibodies can be generated against any of SEQ ID NOS:1 (full length PELP1), 3 (C-terminal region), 5 (N-terminal region), 7-13 (PELP1-ER-blocking peptides), or 14 (PELP1 HI mutant), or any portion including smaller constructs comprising epitopic core regions, including wild-type and mutant epitopes such as those disclosed as SEQ ID NOS:7-13 and 20 (PELP1 antibody generating epitope).

A further embodiment of the present invention encompasses methods of screening potential modulators of PELP1 activity. An example of a screening method includes providing candidate molecules; admixing the candidate molecules with an isolated compound, cell, or experimental animal; measuring one or more characteristics of the compound, cell, or experimental animal; and measuring the effect of the candidate molecule on the one or more characteristics. Measurable characteristics include but are not limited to cell proliferation rate, PELP1 localization, PELP1-ER interactions, or PELP1-chromatin interactions. Assays can be conducted in cell free systems, isolated cells, or organisms including transgenic animals.

In certain embodiments of the invention, the polypeptides, oligopeptides, peptidomimetics, nucleic acids, siRNAs or antibodies are contained in, or combined with, pharmaceutically acceptable carriers to provide a pharmaceutical composition. The active compounds can also be administered by any route recognized as useful by one of skill in the art including parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent. the growth of microorganisms, or other excipients to confer desirable properties on the preparations.

A further embodiment of the present invention relates to therapeutic or diagnostic kits for treatment or diagnosis of disorders related to the estrogen-receptor pathway, particularly cancer and other tumorigenic disorders in estrogen-receptive cells. Such kits comprise one or more therapeutic or diagnostic components packaged for commercial sale.

### BRIEF DESCRIPTION OF THE FIGURES

The following figures form part of the present specification and are included to further demonstrate certain aspects of the present invention. The invention may be better understood by reference to one or more of these drawings in combination with the summary of the invention and the detailed description of specific embodiments presented herein.

FIG. 1A is a Chromatin immunoprecipitation analysis showing basal and dynamic association of PELP1 with chromatin in the presence of estrogen.

FIG. 1B is an assay of PELP1 histone H1 binding by the Far-Western method.

FIG. 1C is a diagram of PELP1 wild type and PELP1 H1 mutant.

FIG. 1D is a polyacrylamide gel of PELP1 wild type and the PELP1 (1-877) deletion mutant PELP1 H1MT.

FIG. 1E is a bar graph showing the down regulation of estrogen mediated induction of reporter gene activation by expression of a PELP1 H1 mutant (PELP1 H1MT) as determined by ERE reporter gene assays.

FIG. 2 shows the effect of PELP (aa 1-877) H1 mutant on estrogen mediated reporter gene activity in breast (MCF-7), endometrial (Ishikawa), cervical (HeLa) and bone (SaoS2) cancer cell lines.

FIG. 3 shows the effect of the PELP1 H1 mutant in blocking estrogen mediated reporter gene activity compared with the effects of certain commonly used anti-estrogens.

FIG. 4 is a figure depicting a proposed model for the function of PELP1, though the present invention is not bound by this theoretical diagram.

FIG. 5 shows the effect of PELP1 siRNA on estrogen mediated ERE reporter gene expression in MCF-7 cells.

FIG. 6 shows the summary of immunoreactive staining of ERα, ERβ, and PELP1 in the human endometrium.

FIG. 7 shows results related to finctional interactions of PELP1 with ERα and ERβ in endometrial cells.

FIG. 8 shows PELP1 interaction with histones H1 and H3.

FIG. 9 shows the effect of a PELP1 mutant lacking a C-terminal histone-binding region on E2-mediated transactivation.

FIG. 10 shows the effects of PELP1 on tamoxifen resistance.

### DETAILED DESCRIPTION

Polypeptides, Oligopeptides, and Peptidomimetics

Blocking or disrupting the activities of PELP1, particularly blocking PELP1 binding with Histone H1 or PELP1-ER interactions, provide a novel pathway for therapeutic activity to slow or halt progression of tumorigenesis and cancer in estrogen-responsive cell types. PELP1 activity can be disrupted by administration of polypeptides, oligopeptides, or peptidomirnetics corresponding to one or more portions of the PELP1 amino acid sequence, particularly the 253 C-terminal amino acids or one of the various ER-interacting regions of the N-terminal portion of the PELP1 polypeptide. Such polypeptides, oligopeptides, or peptidomimetics correspond to all or a portion of SEQ ID NOS:1 (full length PELP1), 3 (C-terminal region), 5 (N-terminal region), 7-13 (PELP1-ER-blocking peptides), or 14 (PELP1 HI mutant).

One embodiment of the present invention relates to compositions comprising one or more polypeptides, fragments, oligopeptides, or peptidomimetics in various forms capable of disrupting PELP1 activity, including those that are naturally occurring or produced through various techniques such as procedures involving recombinant DNA technology or chemical synthesis. Such forms include, but are not limited to, derivatives, variants, and oligomers, as well as fusion proteins or fragments thereof.

Polypeptide variants of the invention include polypeptides that are substantially homologous to the native form, but which have an amino acid sequence different from that of the native form because of one or more deletions, insertions or substitutions. A given amino acid can be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Peptidomimetics of the present invention mimic primary, secondary, or tertiary structures of SEQ ID NOS:1 (full length PELP1), 3 (C-terminal region), 5 (N-terminal region), 7-13 (PELP1-ER-blocking peptides), or 14 (PELP1 HI mutant), or fragments or variants thereof. Preferred peptidomimetics are protease-resistant. Peptidomimetics of the invention include azapeptides, oligocarbamates, oligoureas, β-peptides, γ-peptides, oligo(phenylene ethynylene)s, vinylogous sulfonopeptides, and poly-N-substituted glycines (peptoids). Peptidomimetics of the present invention can be designed and chemically synthesized using techniques well known in the art, such as those outlined in Peptidomimetics Protocols (Methods in Molecular Medicine, V. 23), Kazmierski, W. M., ed., Humana Press (1999).

Nucleic Acids

An alternative embodiment of the present invention is an isolated nucleic acid molecule encoding any of the polypeptides disclosed herein including SEQ ID NOS:2 (full length PELP1), 4 (C-terminal region), 6 (N-terminal region), or 15 (PELP1 H1 mutant), or fragments or variants thereof including complementary sequences. The isolated nucleic acid sequences can be naturally occurring nucleic acid sequences derived from the N- or C-terminal region of the PELP1 gene or variations of such sequences that encode the disclosed polypeptides, including variants caused by redundancies in the genetic code. Alternatively, such nucleic acids can be chemically synthesized based on the desired amino acid code to be expressed. Isolated nucleic acids according to the invention are preferably contained in vectors, including expression vectors capable of directing expression of the polypeptide in an appropriate host cell. Isolated nucleic acids of the present invention can also be included in cell-specific gene therapy compositions. Gene therapy according to the present invention involves providing a nucleic acid encoding a polypeptide to the cell. The polypeptide is then synthesized by the transcriptional and translational machinery of the cell, as well as any that can be provided by the expression construct. In providing antisense, ribozymes, siRNAs and other inhibitors, the method also provides a nucleic acid encoding the inhibitory construct to the cell. All such approaches are herein encompassed within the term “gene therapy”.

Also included in the invention are siRNA molecules. siRNA refers to small interfering RNAs including short hairpin RNAs (shRNAs) (Paddison et al., Genes and Dev., 16:948-58, 2002) capable of causing interference and possibly post-transcriptional silencing of specific genes in cells. RNA interference, including methods of making interfering RNAs, is described and discussed in Bass, Nature, 411:428-29, 2001; Elbashir et al., Nature, 411:494-98, 2001; and Fire et al., Nature, 391:806-11, 1998. siRNAs of the present invention are typically less than 100 base pairs (bp) in length and more preferably are about 20-30 bp or shorter in length. siRNAs of the present invention preferably have one to six nucleotide leaders or tails. Four siRNAs capable of disrupting PELP1 activity have been isolated and are included as SEQ ID NOS:16-19. siRNAs of the present invention can be delivered in the form of naked oligonucleotides, sense or antisense nucleic acid molecules, vectors where the siRNA molecule interacts with the PELP1 gene or its transcripts, or any other means known to those of skill in the art. Interaction of any of the siRNA molecules of the invention causes post-transcriptional silencing or reduced activity of the PELP1 gene in a mammalian cell, including a human cell.

In certain embodiments of the invention, the nucleic acid encoding the PELP1 gene, modulators of the PELP1 gene, or useful fragments thereof can be stably integrated into the genome of the cell. In yet further embodiments, the nucleic acid can be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed and the cell into which the construct is being transformed. Persons of skill in the art routinely select delivery constructs and cells lines and types to meet their needs and can readily optimize such systems for use with the various embodiments of the present invention.

The ability of certain viruses to infect cells or enter cells via receptor-mediated endocytosis, and to integrate into host cell genome and express viral genes stably and efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and in the range of cells they infect, these viruses have been demonstrated to successfully effect gene expression. Adenoviruses do not integrate their genetic material into the host genome, however, and therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation, though any effective means of purifying the vector known in the art can be used.

Additional methods of delivering nucleic acids to cells include liposome-mediated or receptor mediated transfection. In a further embodiment of the invention, the expression construct can be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Still further expression constructs that can be employed to deliver the disclosed nucleic acid construct to target cells are receptor-mediated delivery vehicles. These take advantage of the selective uptake of macromolecules by receptor-mediated endocytosis that will be occurring in the target cells. In view of the cell type-specific distribution of various receptors, this delivery method adds another degree of specificity to the present invention. Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer. In certain aspects of the present invention, the ligand will be chosen to correspond to a receptor specifically expressed on estrogen-responsive target cells such as cells of the testis, ovary and uterine tissues, mammary gland, brain, skeletal muscle, and lung tissues.

In other embodiments, the DNA delivery vehicle component of a cell-specific gene targeting vehicle can comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell.

Antibodies

In certain embodiments, the present invention provides antibodies that bind with high specificity to the PELP1 polypeptide. In addition to antibodies generated against the full length N- or C-termiinal region, antibodies can also be generated in response to smaller constructs comprising epitopic core regions, including wild-type and mutant epitopes. An example of one such epitope is disclosed as SEQ ID NO:20. This epitope has been used to generate antibodies against PELP1 in rabbits. Additional epitopes can be derived from any of the amino acid sequences disclosed herein. Further, the polypeptides, fragments, variants, fusion proteins, etc., as set forth above can be employed as “immunogens” in producing antibodies immunoreactive therewith. Generally, IgG or IgM alone or in combination are preferred because they are the. most common antibodies in the physiological situation and because they are most easily made in a laboratory setting, though any antibody type can be used.

These antigenic determinants or epitopes can be either linear or conformational (discontinuous). Linear epitopes are composed of a single section of amino acids of the PELP1 polypeptide, while conformational or discontinuous epitopes are composed of amino acids sections from different regions of the polypeptide chain that are brought into close proximity upon protein folding (C. A. Janeway, Jr. and P. Travers, Immuno Biology 3:9 (Garland Publishing Inc., 2nd ed. 1996)). Epitopes can be identified by any of the methods known in the art.

Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

Both polyclonal and monoclonal antibodies elicited by the epitopes of the polypeptides of the invention, whether such epitopes have been isolated or remain part of the polypeptides, can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit, chicken or any other species origin known to one of skill in the art. Due to the ease of preparation and ready availability of reagents, murine monoclonal antibodies will often be preferred. “Humanized” antibodies are also contemplated, however, as are chimeric antibodies from mouse, rat, or other species, bearing human constant or variable region domains alone or in combination, bispecific antibodies, recombinant and engineered antibodies and fragments thereof. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993). Procedures to generate antibodies transgenically can be found in GB 2,272,440, U.S. Pat. Nos. 5,569,825 and 5,545,806 and related patents claiming priority there from, all of which are incorporated by reference herein.

Antigen-binding fragments of the antibodies, which can be produced by conventional techniques, are also encompassed by the present invention. Examples of such fragments include, but are not limited to, Fab′, Fab, F(ab′)2, single domain antibodies (DABS), Fv, scFv (single chain Fv), and the like. Antibody fragments and derivatives produced by genetic engineering techniques are also provided.

The present invention further provides antibodies against PELP1 proteins, polypeptides or peptides, generally of the monoclonal type, that are linked to one or more other agents to form an antibody conjugate. Any antibody of sufficient selectivity, specificity and affinity can be employed as the basis for an antibody conjugate. Such properties can be evaluated using conventional immunological screening methodology known to those of skill in the art.

Certain examples of antibody conjugates are those conjugates in which the antibody is linked to a detectable label. “Detectable labels” are compounds or elements that can be detected due to their specific functional properties, or chemical characteristics, the use of which allows the antibody to which they are attached to be detected, and further quantified if desired. Another such example is the formation of a conjugate comprising an antibody linked to a cytotoxic or anti-cellular agent, as may be termed “immunotoxins”

Antibody conjugates are thus preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging”.

It also is possible to use antibodies to ascertain the structure of a target compound activator or inhibitor. This approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate polypeptides from banks of chemically- or biologically-produced polypeptides. Selected polypeptides would then serve as the pharmacore. Anti-idiotypes can be generated using the methods described herein for producing antibodies, using an antibody as the antigen.

Pharmaceuticals

In certain embodiments of the invention, the polypeptides, peptidomimetics, nucleic acids or antibodies are contained in, or combined with pharmaceutically acceptable carriers. The active compounds can also be administered by any route known in the art including parenteral, intraperitoneal, subcutaneous, intravenous, intramuscular, sublingual, inhaled, oral and the like. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. These preparations often contain a preservative to prevent the growth of microorganisms during storage. Methods of selecting usefull and desired carriers or excipients, alone or in combination, are well known to those of skill in the art.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The pharmaceutical forms suitable for injectable use include sterile aqueous solutions or dispersions and sterile powders for the extemporaneous preparation of sterile injectable solutions or dispersions. In all cases the form must be sterile and must be suitably fluid. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus ny additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Screening

The present invention also contemplates the screening of compounds for their ability to modulate PELP1. Screening methods can be conducted in cell free systems, isolated cells, or organisms including transgenic animals. An example of a cell free screening method includes providing candidate molecules; admixing the candidate molecules with an isolated compound, cell, or experimental animal; measuring one or more characteristics of the compound, cell, or experimental animal; and measuring the effect of the candidate molecule on the one or more characteristics. Measurable characteristics include but are not limited to cell proliferation rate, PELP1 localization, PELP1-ER interactions, or PELP1-chromatin interactions. in cells.

Various cell lines can be used for isolated cell assays including but not limited to MCF-7, T47D, MDA MB-231 and ZR75R human breast cancer cells, SAOS-2, HepG2, Caco-2 cells, U2OS bone cells, Ishikawa, RL 95-2, SW1748, HEC1A, HEC1B, endometrial cells, He La, or cells specifically engineered for this purpose, including but not limited to MCF-7-PELP1 cells, MCF-7-PELP1 H1 mutant cells, Ishikawa-PELP1 wild type cells, Ishikawa-PELP1-H1 mutant cells, or PELP1-Teton wild type inducible cells, PELP1 NLS mutant cells can be utilized for such screening assays.

Depending on the assay, culture may be required. The cell is examined using any of a number of different physiologic assays. Alternatively, molecular analysis can be performed, for example, looking at protein expression, mRNA expression (including differential display of whole cell or polyA RNA) and others. Additional screening methods and construction of screening protocols are well known to those of skill in the art and are useful in the present invention.

Therapeutic or Diagnostic Kits

Therapeutic or diagnostic kits of the present invention are kits comprising at least one modulator of PELP1 including but not limited to, protein, polypeptide, peptide, peptidomimetic, inhibitor, gene, vector, antibody, antibody conjugate or other effector in a pharmaceutically acceptable formulation supplied in a suitable container. The kit can also comprise any of the PELP1 modulators of the invention together with a traditional SERM such as tamoxifen or other selective estrogen-receptor modulators known in the art. The kit can have a single container or it can have distinct containers for each of variously supplied compounds to comprise the complete kit.

When the components of the kit are provided in one or more liquid solutions, the liquid solution is an aqueous solution, with a sterile aqueous solution being particularly preferred. The compositions of PELP1 modulator or pharmaceutically acceptable salts thereof can also be formulated into a syringeable composition. In which case, the container can be a syringe, pipette, or other such like apparatus, from which the formulation can be applied to an infected area of the body, injected into an animal, or even applied to or mixed with other components of the kit.

Components of the kit can also be provided as dried powder(s). When components are provided as a dry powder, the powder can be reconstituted by the addition of a suitable solvent. It is envisioned that the solvent can be provided in a separate container as part of the kit.

The following Examples are offered for the purpose of illustrating the present invention and are not to be construed to limit the scope of this invention. The contents of all references, patents and published patent applications cited throughout this application are hereby incorporated herein by reference.

### EXAMPLES

### Example 1

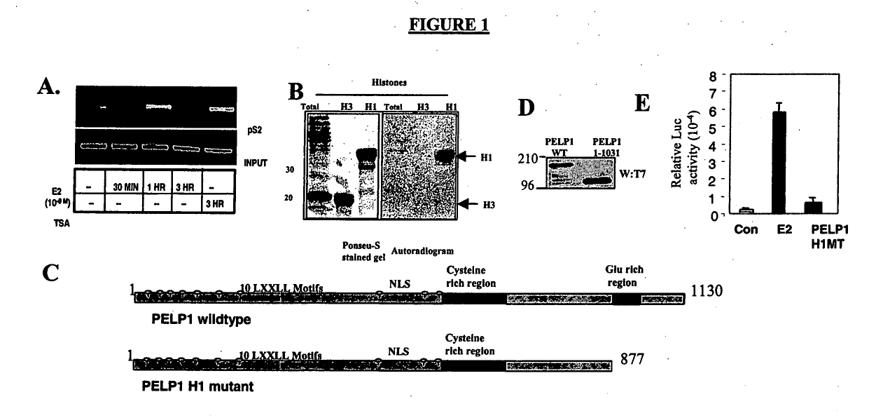

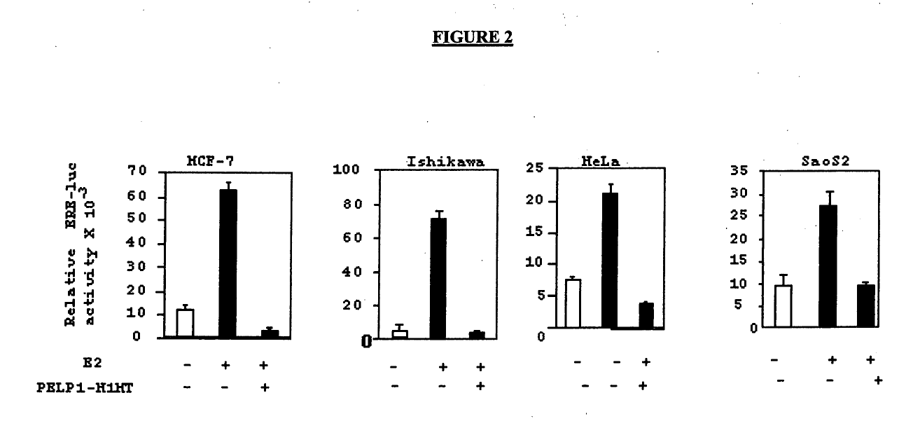

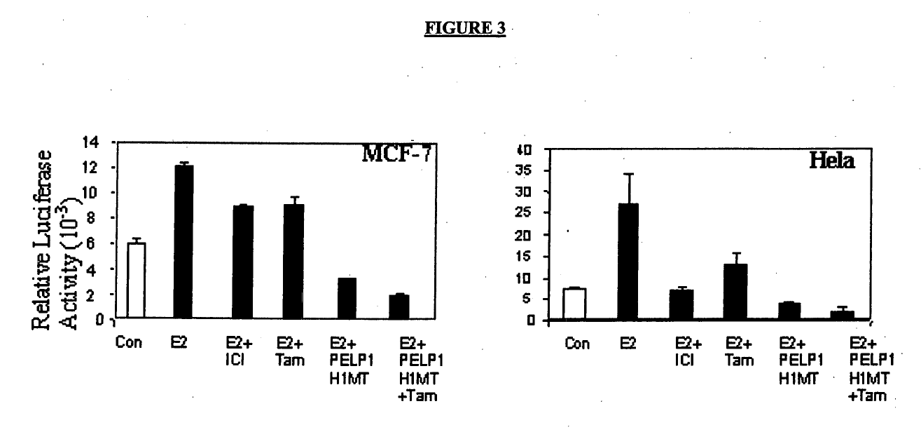

Characterization of Histone H1 binding of a PELP1 H1 mutant. Experiments were performed to demonstrate the presence of a histone binding domain in PELP1. These experiments examine whether PELP1 is recruited to the chromatin and whether it interacts with Histone H1. FIG. 1A shows the results of a CHIP analysis in which a PELP1 stable clone was grown in charcoal stripped serum for two days, treated with or without E2 for periods of 30 minutes, 1 hour or 3 hours or with TSA for 3 hours. T7-PELP1 was immunoprecipitated with anti-T7 antibody, bound chromatin was eluted and PCR amplification primers specific to the pS2 gene (−359 to −30) were used in the CHIP analysis. The CHIP analysis showed basal accumulation of PELP1 in the absence of estrogen stimulation. The 30-minute treatment showed no PELP1 association, followed by increased recruitment after 60 minutes of E2 treatment. Continuation of E2 treatment for 3 hours resulted in complete loss of PELP1 from the pS2 promoter. These results suggest that PELP1 is recruited to E2 responsive promoters in a dynamic manner and deacetylase complexes may have a role in the recruitment of PELP1 to the pS2 promoter.

The results of the Far-Western assay depicted in FIG. 1B show that PELP1 interacts with Histone H1. Native histones were purified from MCF-7 cells and run on a 15% SDS-PAGE gel along with purified H3 or H1 histones (Roche Biochemicals). The gel was transferred to nitrocellulose and 35S-labelled PELP1 was generated using an in vitro transcription and translation system for use as a probe. PELP1 interacting bands were identified by autoradiography. The results of this experiment indicate that PELP1 can interact specifically with histone H1 both as a component of total histones and as purified histone H1. No binding was observed to other histones or to purified histone H3.

FIG. 1C is a diagram comparing the structure of the PELP1 H1 mutant prepared by the inventors with the structure of wildtype PELP1. The mutant was constructed by means of site directed mutagenesis wherein a stop codon was introduced after the codon for amino acid 877 of wildtype PELP1. The location of the consensus nuclear localization motif is indicated by the abbreviation “NLS” in the diagram.

FIG. 1D shows expression of the PELP1 H1 mutant versus wildtype PELP1. The deletion of the C-terminal region results in expression of a smaller protein as shown by transient transfection assay followed by Western analysis.

The expression of the PELP1 H1 mutant lacking the Histone H1 binding domain results in a blocking of estrogen-mediated transcriptional activation. The functionality of the PELP1 mutant was demonstrated in the ERE reporter gene assays depicted in FIG. 1E. Ishikawa (human endometrial adenocarcinoma) cells were transfected with ERE reporter gene with or without the PELP1 H1 mutant. The cells were then treated or not treated with estrogen and the reporter gene activity was measured.

### Example 2

Characterization of a PELP1 dominant negative mutant. Breast cancer cell line MCF-7, endometrial cancer cell line Ishikawa, cervical cancer cell line HeLa and osteosarcoma SaoS2 cells were transfected with an ERE luciferase reporter with or without the dominant-negative PELP1 H1 mutant. After 24 hours, cells were treated with or without E2 (10^{-9M})and 24 hours later luciferase reporter activity was measured. The results of the assay are shown in FIG. 2. In all the four model cells tested, addition of estrogen stimulated transcription from its reporter gene several fold. Expression of the Histone H1 mutant, however, substantially reduced the magnitude of the transcriptional activation by estrogen. These results indicate that the C-terminal region or PELP1 contains a Histone H1 binding region that is important for maintenance of normal estrogen-mediated transcriptional functions. The interaction of PELP1 and histone H1 is surprising because PELP1 shares little homology with other NR co-regulator proteins.

Expression of the PELP1 H1 mutant therefore effectively suppressed the estrogen mediated ER coactivation functions in breast, osteosarcoma and endometrial cancer cells.

### Example 3

Comparison of the effect of the PELP1 H1 mutant versus certain commonly-used anti-estrogens. MCF-7 cells or Hela cells were transfected with an ER responsive reporter (ERE-luciferase). Some cells were transfected with the PELP1 H1 mutant and some were not. The cells were further treated with estrogen, with estrogen in the presence of ICI182780, with estrogen in the presence of Tamoxifen, with Tamoxifen plus the PELP1 H1 mutant, or with Tamoxifen plus estrogen plus the PELP1 H1 mutant, as shown in FIG. 3.

In both the MCF-7 and Hela cell lines, estrogen stimulated the ERE reporter gene and the anti-estrogens ICI and tamoxifen reduced the magnitude of the ERE activity, as shown in the bar graph in FIG. 3. PELP1 H1 mutant significantly also blocked E2 mediated reporter activity and was much more potent than ICI or Tamoxifen. In addition, combining tamoxifen with the PELP1 H1 mutant produced a much more significant inhibition than one agent or combination of agents tested.

### Example 4

Deregulation of PELP1 in tumor cell lines. PELP1 expression was studied in a variety of cell lines. Both tamoxifen-sensitive and tamoxifen-resistant cells expressed similar levels of PELP1. PELP1 was, however, found to be differently localized in. tamoxifen-resistant cells. Immunohistological examination of PELP1 expression in tumor cells indicated that PELP1 is primarily localized in the cytoplasm of the tumor cells, versus its being localized in the nucleus in normal cells. Deregulation of PELP1 expression was observed in both breast and endometrial tumors.

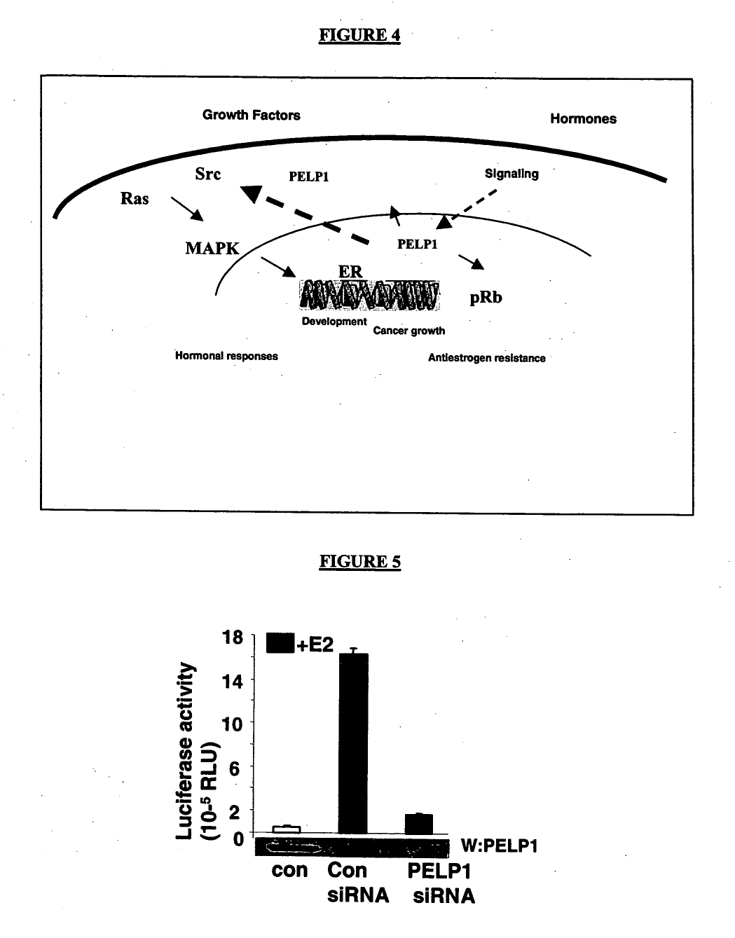

Not to be bound by theory, altered localization of PELP1 in cancerous cell lines and the ability of PELP1 to modulate the activity of SERMs are believed to indicate that PELP1 plays a role in tamoxifen and hormonal resistance through the mechanism of activation of non-genomic signaling by PELP1. A model for a proposed mechanism for PELP1 is depicted in FIG. 4.

Under normal physiological conditions PELP1 localizes to the nuclear compartment. Estrogen enhances PELP1 interactions with ER and pRb potentiating ER mediated genomic responses (Classical genomic pathway). In pathological conditions such as breast cancer, PELP1 localization is altered and PELP1 predominantly localizes in the cytoplasm. When PELP1 is present in the cytoplasm, estrogen enhances the PELP1-ER and PELP1-src kinase interactions. These enhanced interactions eventually lead to activation of the MAPK pathway (non-genomic pathway) and increased phosphorylation of ER resulting in altered hormonal responses to antiestrogens such as tamoxifen, thus contributing to resistance to the effects of tamoxifen.

### Example 5

Interference with PELP1 function blocks tamoxifen-mediated agonist activity in endometrial cell lines. Disruption of PELP1 functions was examined to assess interference with tamoxifen-mediated agonist signaling using reporter gene assays and cell growth assays performed in an endometrial cell line (Ishikawa) and a breast cancer cell line (MCF-7). Cancer cells were co-transfected with ERE reporter gene along with either (a) PELP1 cDNA, (b) PELP1 H1 mutant cDNA (aa 1-877), (c) PELP1 mutant cDNA lacking the nuclear localization signal (PELP-NLS mutant), or (d) PELP1-specific siRNA. Cells were stimulated with estrogen for two days and reporter gene activity and cell number were measured. Over-expression of wild-type PELP1 augmented tamoxifen-mediated agonist activity in Ishikawa cells but not in MCF-7 cells. Interestingly, expression of the PELP1-NLS mutant, which predominantly localizes in the cytoplasm, enhanced tamoxifen-mediated agonist signaling in both MCF-7 and Ishikawa cells. Expression of the PELP1 H1 mutant (1-877) disrupted tamoxifen-mediated agonist activity in Ishikawa cells. Furthermore, expression of PELP1-specific siRNA also disrupted tamoxifen-mediated agonist activity in Ishikawa cells.

### Example 6

Treatment of cells with PELP1 siRNA substantially reduced PELP1 expression levels. MCF-7 cells were transfected with an ERE reporter gene along with either a control siRNA or with a cocktail of four PELP1 siRNAs. Cells were treated with or without estrogen and reporter activity was measured. Cell lysates were also analyzed by Western blotting to examine the level of PELP1.

As shown in FIG. 5, treatment of MCF-7 cells with PELP1 siRNA substantially reduced expression of PELP1. Down-regulation of PELP1 using siRNA also significantly affected the ER mediated trans-activation functions to levels similar to those observed in cells expressing the PELP1 H1 mutant. These results show that PELP1 siRNAs can provide alternative means of manipulating signaling in cancer cells similar to the earlier described methods involving PELP1 polypeptides and mutants.

### Example 7

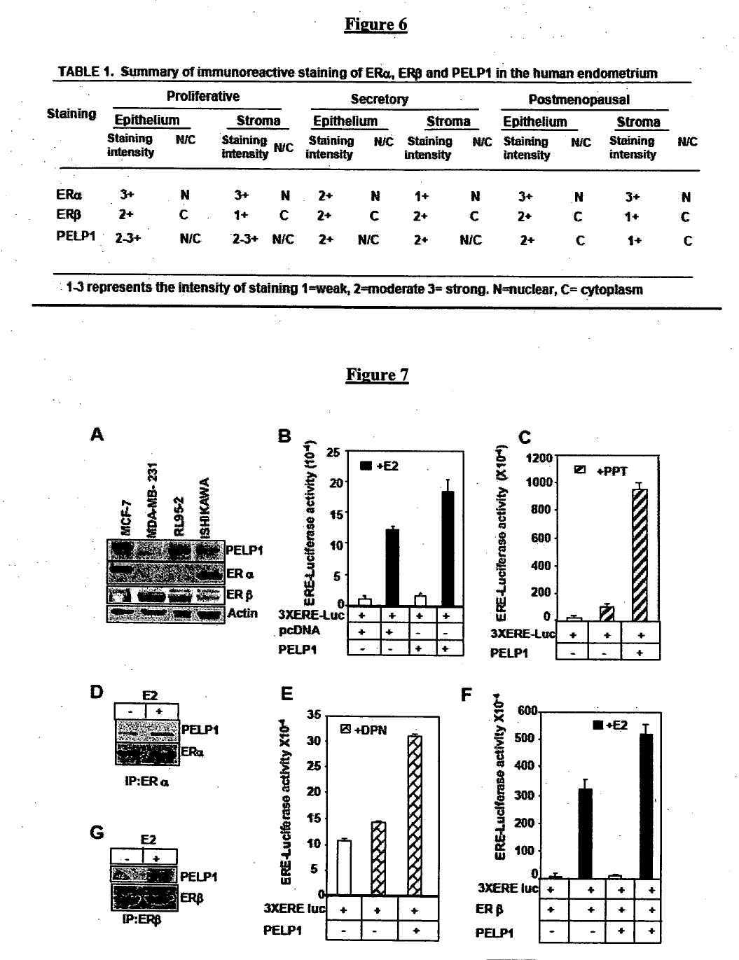

PELP1 expression and localization in normal and cancerous endometrial cells. The expression and localization of PELP1 was characterized in both normal and cancerous endometrium. FIG. 6 shows that while PELP1 is expressed in all stages of endometrium, this protein exhibits distinct localization depending on the phase.

PELP1 is widely expressed in endometrial cancer cells including the widely used endometrial cell lines (Ishikawa and RL 95-2). Control MCF-7 (ERα-positive) and MDA-MB-231 (ERβ-positive) breast cancer cells were also analyzed. (FIG. 7A). ER transactivation assays using ER-positive Ishikawa cells as a model system illustrate that coexpression of PELP1 increased ERE-luciferase (luc) activity in ligand-stimulated cells by 9 fold compared to 6 fold observed in vector-transfected cells, suggesting that PELP1 also acts as a coactivator of ER in endometrial cells (FIG. 7B). PELP1 modulation of the transactivation functions of both ER subtypes was performed using ERα or ERβ specific ligands. When PELP1-transfected cells were treated with PPT, the ERα specific ligand, 3XEREluc reporter activity was increased 9 times more than the vector-transfected control (FIG. 7C), suggesting that PELP1 coactivates ERα-dependent transcription and cooperates with the endogenous ERα and its specific ligand PPT. In consonance with these results, treatment of Ishikawa cells with E2 resulted in an enhanced association of PELP1 with ERα in vivo (FIG. 7D). As shown in FIG. 7E, treatment of Ishikawa cells with an ERβ specific ligand DPN also stimulated ERE-luc activity, though only three times more than seen in control cells, suggesting that PELP1 can also cooperate with the transcriptional activity of ERβ. PELP1 also promotes ERβ transcriptional activity in Ishikawa cells when cotransfected with PELP1 (FIG. 7F). Further, endogenous PELP1 effectively interacts with ERβ in Ishikawa cells in a ligand-dependent manner (FIG. 7G). Collectively these results suggest that PELP1 acts as a coactivator of both ER subtypes, however PELP1 exhibited more magnitude of coactivation with ERα compared to ERβ in Ishikawa cells.

### Example 8

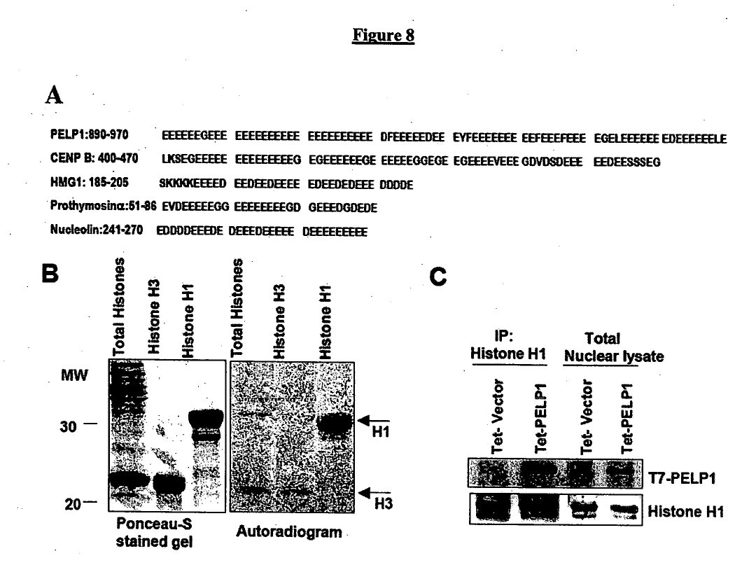

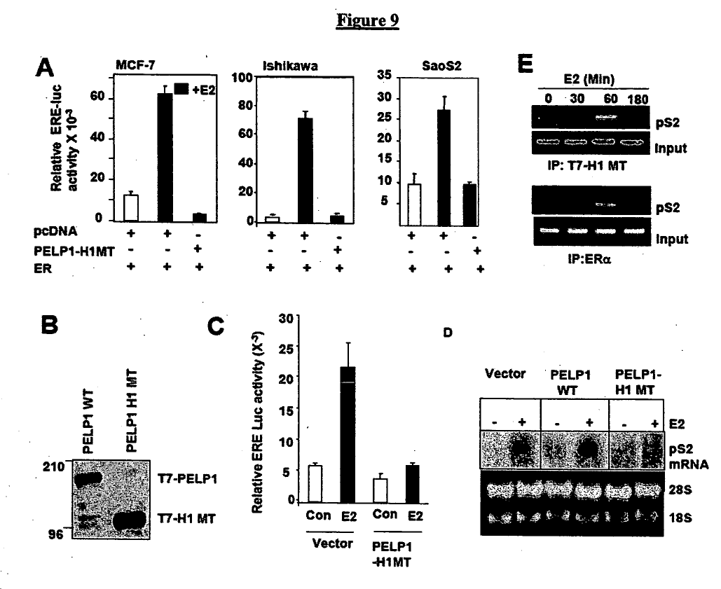

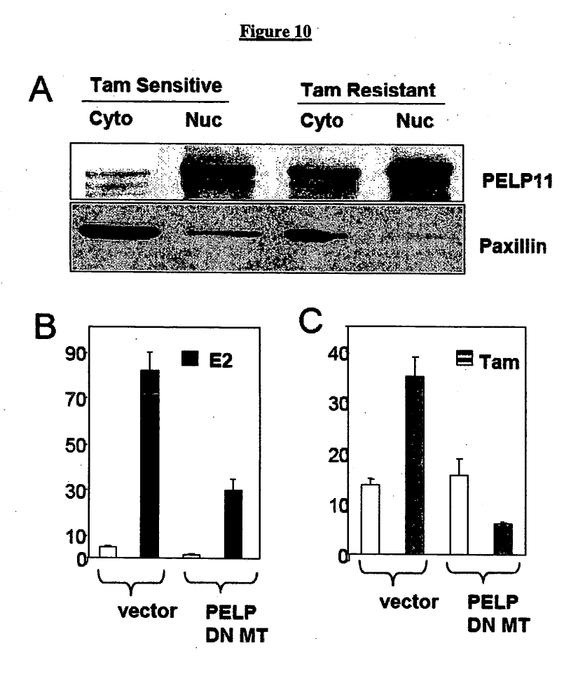

PELP1 interactions with histones. Biochemical and scanning confocal microscopic analysis are used to demonstrate nuclear localization and functional implications of PELP1. Subnuclear fractionation showed PELP1 association with chromatin and nuclear matrix fractions. Ligand stimulation promoted recruitment of PELP1 to 17-β-estradiol (E2) responsive promoters, its co-localization with acetylated H3, and increased PELP1-associated histone acetyltransferase enzymatic activity. Far western analysis revealed that PELP1 interacts with histones 1 and 3 (H1 and H3), with more preference towards H1. (FIG. 8). Using deletion analysis, the PELP1 C-terminal region has been identified as the H1 binding site. A PELP1 mutant lacking H1 binding domain acts as a dominant negative and blocks ERα mediated transcription. (FIG. 9). Chromatin immunoprecipitation analysis shows a cyclic association and dissociation of PELP1 with the promoter, with recruitment of H1, and PELP1 occurring in opposite phases. PELP1 overexpression increases the micrococal nuclease sensitivity of estrogen response element-containing nucleosomes. These results suggest that PELP1 participates in chromatin remodeling activity via displacement of H1 in cancer cells.

### Example 9

The role of PELP1 in tamoxifen resistence. The localization of PELP1 in 60 breast cancer specimens was analyzed by immunohistochemistry. To examine the functional consequences of altetred localization of ER coactivator PELP1, MCF-7 model cells which specifically expresses PELP1 in the cytoplasm (PELP1-cyto) were generated. Reporter gene assays, protein, confocal and cell biology based methods were used on MCF-7, MCF-7-PELP1 wild type, and MCF7-PELP1-cyto model cells to show that tamoxifen sensitivity is affected by localization of PELP1. (FIG. 10). Immunohistological examination of PELP1 in 60 breast tumor specimens suggested that it is predominantly localized in the cytoplasm as opposed to nuclear localization in the normal tissues. PELP1-cyto cells conferred hypersensitivity to estrogen and exhibited resistance to tamoxifen. PELP1-cyto cells also exhibited excessive MAPK activation upon E2 treatment and constitutive PI3K activity. Further, PELP1-cyto cells exhibited constitutive association of PELP1 with the p85 subunit of PI3K. PELP1 cyto cells also exhibited increased phosphorylation of ER on Ser 118 and ser 167. These results suggest that altered localization of coactivators such as PELP1 which has potential to activate nongenomic signaling pathways such as MAPK and PI3K and thus enhance ER phosphorylation and which lead to tamoxifen agonist actions and such actions may lead to tamoxifen resistance.

The foregoing descriptions of the invention are intended merely to be illustrative thereof and other embodiments, modifications, and equivalents of the invention are within the scope of the invention recited in the claims appended hereto. It should be appreciated by those skilled in the art that the conception and the specific embodiments disclosed can be readily utilized as a basis for modifying or designing other compositions or methods for carrying out the same purposes of the present invention. It should also be realized by those skilled in the art that such equivalent compositions or methods do not depart from the spirit and scope of the invention as set forth in the appended claims.

### REFERENCES

1. Kliewer, S. A., Lehmann, J. M., and Willson, T. M. (1999) Science 284, 757-760
2. Xu, L., Glass, C. K., and Rosenfeld, M. G. (1999) Curr. Opin. Genet. Dev. 9, 140-147
3. McKenna, N. J., Lanz, R. B., and O'Malley, B. W. (1999) Endocr. Rev. 20, 321-344
4. Tsai, M. J., and O'Malley, B. W. (1994) Annu. Rev. Biochem. 63, 451-486
5. Glass, C. K, Rose, D. W., and Rosenfeld, M. G. (1997) Curr. Opin. Cell Biol. 9, 222-232
6. Ma, H., Hong, H., Huang, S. M., Irvine, R. A., Webb, P., Kuahner, P. J., Coetzee, G. A., and Stallcup, M. R. (1999) Mol. Cell. Biol. 19, 6164-6173
7. Torchia, J., Glass, C., and Rosenfeld, M. G. (1998) Curr. Opin. Cell Biol. 10, 373-383
8. Chakravarti, D., LaMorte, V. J., Nelson, M. C., Nakajima, T., Schulman, I. G., Juguilon, H., Montminy, M. and Evans, R. M. (1996) Nature 383, 99-103
9. Wagner, B. L., Norris, J. D., Knotts, T. A., Weigel, N. L., and McDonnell, D. P. (1998) Mol. Cell. Biol. 18, 1369-1378
10. Huang, S. M., and Stallcup, M. R. (2000) Mol. Cell. Biol. 20, 1855-1867
11. Huang, N., Vom, B., Garnier, J. M., Lerouge, T., Vonesch, J. L., Lutz, Y., Chambon, P., and Losson, R. (1998) EMBO J. 17, 3398-3412
12. Cavailles, V., Dauvois, S., L'Horset, F., Lopez, G., Hoare, S., Kushner, P. J., and Parker, M. G. (1995) EMBO J. 14, 8741-3751
13. Heery, D. 14., Kalkhoven, E., Hoare, S., and Parker, M. G. (1997) Nature 387, 733-736
14. Spencer, T. E., Jenster, G., Burcin, M. M., Allis, C. D., Zhou, J., Mizzen, C. A., McKenna, N. J., Onate, S. A., Tsai, S. Y., Tsai, M. J., and O'Malley, B. W. (1997) Nature 389, 194-198
15. Nagy, L., Kao, H Y., Chakravarti, D., Lin, R J., Hassig, C. A., Ayer, D. E., Schreiber, S. L, and Evans, R. M. (1997) Cell 89, 373-380
16. Glass, C. K, and Rosenfeld, M. G. (2000) Genes Dev. 14, 121-141
17. Couse, J. F., and Korach, K S. (1999) Endocr. Rev. 20, 358-417
18. Warner, M., Nilsson, S., and Gustafsson, J. A. (1999) Curr. Opin. Obstet. Gynecol. 11, 249-254
19. Kumar, V., Green, S., Stack, G., Berry, M., Jin, J. R., and Chambon, P. (1987) Cell 51, 941-951
20. Dubik, D., and Shiu, 1%. P. (1988) J. Biol. Chem. 263, 12705-12708
21. Zhang, J., Thomsen, J., Johansson, J., Gustafsson, J., and Treuter, E. (2000) J. Biol. Chem. 275, 39855-39859
22. Tcherepanova, I., Puigserver, P., Norris, J. D., Spiegelman, B. M., and McDonnell, D. P. (2000) J. Biol. Chem. 275, 16302-16308
23. Adam, L, Vadlamudi, R., Kondapaka, S. B., Chernoff 3., Mendelsohn, J., and Kumar, R. (1998) J. Biol. Chem. 273, 28238-28246
24. Joung, I., Strominger, J. L., and Shin, J. (1996) Proc. Natl. Acad. Sci, U.S.A. 93, 5991-5995
25. Adam, L., Vadlamudi, R., Mandal, M., Chernoff, J., and Kumar, R. (2000) J. Biol. Chem. 275, 12041-12050
26. Mazumdar, A., Wang, R. A., Mishra, S. K, Adam, L., Bagheri-Yarmand, R., Mandal, M., Vadlamudi, R. K, and Kumar, R. (2001) Nat. Cell Biol. 3, 30-37
27. Wang, R. A., Nakane, P. K, and Koji, T. (1998) Biol. Reprod. 58, 1250-1256
28. Chelsky, D., Ralph, R., and Jonak, G. (1989) Mol. Cell. Biol. 9, 2487-2492
29. Mermod, N., O'Neill, E. A., Kelly, T. J., and Tjian, R. (1989) Cell 58, 741-753
30. Fan, S., Wang, J., Yuan, R., Ma, Y., Meng, Q., Erdos, M. R., Pestell, R. G., Yuan, F., Aubom, K. J., Goldberg, I. D., and Rosen, E. M. (1999) Science 284, 1354-1356
31. Krumm, A., Madisen L., Yang, X., Goodman, R., Nakatani, Y., and Groudine, M. (1998) Proc. Natl. Acad. Sci. U.S.A. 95, 14501-14506
32. Brady. M. E., Ozanne, D. 14., Gaughan, L, Waite, I., Cook, S., Neal, D. E., and Robson, C. N. (1999) J. Biol. Chem. 274, 17599-17604
33. Horwitz, K B., Jackson, T. A., Bain, D. L., Richer, J. K., Takimoto, G. S., and Tung, L. (1996) Mol. Endocrinol. 10, 1167-1177
34. Feng, W., Ribeiro, R. C., Wagner, R. L, Nguyen, H., Apriletti, J. W., Fletterick, R. J., Baxter, J. B., Kushner, P. J., and West, B. L (1998) Science 280, 1747-1749
35. Henttu, P. M., Kalkhoven, E., and Parker, M. G. (1997) Mol. Cell. Biol. 17,1832-1839
36. Lee, C. H., and Wei, L. N. (1999) J. Biol. Chem. 274, 31820-31326
37. Lee, C. H., Chinpaisal, C., and Wei, L. N. (1998) Mol. Cell. Biol. 18, 6745-6755
38. Anzick, S. L, Kononen, J., Walker, R. L., Azorsa, D. O., Tanner, M. M., Guan, X. Y., Sauter, G., Kallioniemi, O. P., Trent, J. M., and Meltzer, P. S. (1997) Science 277, 965-968.
39. Setharaman B., and Vadlamudi, R. K. (2003) J. Biol. Chem. 278, 22118-22127.
40. Balasenthil S., Broaddus R., Gustafsson J. A., Kumar R., Vadlamudi R. K. (2003) Proceedings of San Antonio Breast Cancer Symposium, Abstract #27.

41. Mishra S. K., Balasenthil S., Nguyen D., Vadlamudi R. K. (2004) Gene 330, 115-122.

| SEQ ID NO:1 (PELP1 full length polypeptide; GenBank | | | | | |
|---|---|---|---|---|---|
| Accession No. NP_055204) | | | | | |
| MAAAVLSGPS | AGSAAGVPGG | TGGLSAVSSG | PRLRLLLLES | VSGLLQPRTG | SAVAPVHPPN |
| RSAPHLPGLM | CLLRLHGSVG | GAQNLSALGA | LVSLSNARLS | SIKTRFEGLC | LLSLLVGESP |
| TELFQQHCVS | WLRSIQQVLQ | TQDPPATMEL | AVAVLRDLLR | YAAQLPALFR | DISMNHLPGL |
| LTSLLGLRPE | CEQSALEGMK | ACMTYFPRAC | GSLKGKLASF | FLSRVDALSP | QLQQLACECY |
| SRLPSLGAGF | SQGLKHTESW | EQELHSLLAS | LHTLLGALYE | GAETAPVQNE | GPGVEMLLSS |
| EDGDAHVLLQ | LRQRFSGLAR | CLGLMLSSEF | GAPVSVPVQE | ILDFICRTLS | VSSKNISLHG |
| DGPLRLLLLP | SIHLEALDLL | SALILACGSR | LLRFGILIGR | LLPQVLNSWS | IGRDSLSPGQ |
| ERPYSTVRTK | VYAILELWVQ | VCGASAGMLQ | GGASGEALLT | HLLSDISPPA | DALKLRSPRG |
| SPDGSLQTGK | PSAPKKLKLD | VGEAMAPPSH | RKGDSNANSD | VCAAALRGLS | RTILMCGPLI |
| KEETHRRLHD | LVLPLVMGVQ | QGEVLGSSPY | TSSRCRRELY | CLLLALLLAP | SPRCPPPLAC |
| ALQAFSLGQR | EDSLEVSSFC | SEALVTCAAL | THPRVPPLQP | MGPTCPTPAP | VPPPEAPSPF |
| RAPPFHPPGP | MPSVGSMPSA | GPMPSAGPMP | SAGPVPSARP | GPPTTANHLG | LSVPGLVSVP |
| PRLLPGPENH | RAGSNEDPIL | APSGTPPPTI | PPDETFGGRV | PRPAFVHYDK | EEASDVEISL |
| ESDSDDSVVI | VPEGLPPLPP | PPPSGATPPP | IAPTGPPTAS | PPVPAKEEPE | ELPAAPGPLP |
| PPPPPPPPVP | GPVTLPPPQL | VPEGTPGGGG | PPALEEDLTV | ININSSDEEE | EEEEEGEEEE |
| EEEEEEEEDF | EEEEEDEEEY | FEEEEEEEEE | FEEEFEEEEG | ELEEEEEEED | EEEEEELEEV |
| EDLEFGTAGG | EVEEGAPPPP | TLPPALPPPE | SPPKVQPEPE | PEPGLLLEVE | EPGTEEERGA |
| DTAPTLAPEA | LPSQGEVERE | GESPAAGPPP | QELVEEEPSA | PPTLLEEEPE | DGSDKVQPPP |
| ETPAEEEMET | ETEAEALQEK | EQDDTAAMLA | DFIDCPPDDE | KPPPPTEPDS | |
| SEQ ID NO:2 (PELP1 full length nucleic acid, | | | | | |
| GenBank Accession No. NM_014389): | | | | | |
| atggcggcag | ccgttctgag | tgggccctct | gcgggctccg | cggctggggt | tcctggcggg |
| accgggggtc | tctcggcagt | gagctcgggc | ccgcggctcc | gcctgctgct | gctggagagt |
| gtttctggtt | tgctgcaacc | tcgaacgggg | tctgccgttg | ctccggtgca | tcccccaaac |
| cgctcggccc | cacatttgcc | cgggctcatg | tgcctattgc | ggctgcatgg | gtcggtgggc |
| ggggcccaga | acctttcagc | tcttggggca | ttggtgagtc | tcagtaatgc | acgtctcagt |
| tccatcaaaa | ctcggtttga | gggcctgtgt | ctgctgtccc | tgctggtagg | ggagagcccc |
| acagagctat | tccagcagca | ctgtgtgtct | tggcttcgga | gcattcagca | ggtgttacag |
| acccaggacc | cgcctgccac | aatggagctg | gccgtggctg | tcctgaggga | cctcctccga |
| tatgcagccc | agctgcctgc | actgttccgg | gacatctcca | tgaaccacct | ccctggcctt |
| ctcacctccc | tgctgggcct | caggccagag | tgtgagcagt | cagcattgga | aggaatgaag |
| gcttgtatga | cctatttccc | tcgggcttgt | ggttctctca | aaggcaagct | ggcctcattt |
| tttctgtcta | gggtggatgc | cttgagccct | cagctccaac | agttggcctg | tgagtgttat |
| tcccggctgc | cctctttagg | ggctggcttt | tcccaaggcc | tgaagcacac | cgagagctgg |
| gagcaggagc | tacacagtot | gctggcctca | ctgcacaccc | tgctgggggc | cctgtacgag |
| ggagcagaga | ctgctcctgt | gcagaatgaa | ggccctgggg | tggagatgct | gctgtcctca |
| gaagatggtg | atgcccatgt | ccttctccag | cttcggcaga | ggttttcggg | actggcccgc |
| tgcctagggc | tcatgctcag | ctctgagttt | ggagctcccg | tgtccgtccc | tgtgcaggaa |
| atcctggatt | tcatctgccg | gaccctcagc | gtcagtagca | agaatattag | cttgcatgga |
| gatggtcccc | tgcggctgct | gctgctgccc | tctatccacc | ttgaggcctt | ggacctgctg |
| tctgcactca | tcctcgcgtg | tggaagccgg | ctcttgcgct | ttgggatcct | gatcggccgc |
| ctgcttcccc | aggtcctcaa | ttcctggagc | atcggtagag | attccctctc | tccaggccag |
| gagaggcctt | acagcacggt | tcggaccaag | gtgtatgcga | tattagagct | gtgggtgcag |
| gtttgtgggg | cctcggcggg | aatgcttcag | ggaggagcct | ctggagaggc | cctgctcacc |
| cacctgctca | gcgacatctc | cccgccagct | gatgccctta | agctgcgtag | cccgcggggg |
| agccctgatg | ggagtttgca | gactgggaag | cctagcgccc | ccaagaagct | aaagctggat |
| gtgggggaag | ctatggcccc | gccaagccac | cggaaagggg | atagcaatgc | caacagcgac |
| gtgtgtgcgg | ctgcactcag | aggcctcagc | cggaccatcc | tcatgtgtgg | gcctctcatc |
| aaggaggaga | ctcacaggag | actgcatgac | ctggtcctcc | ccctggtcat | gggtgtacag |
| cagggtgagg | tcctaggcag | ctccccgtac | acgagctccc | gctgccgccg | tgaactctac |
| tgcctgctgc | tggcgctgct | gctggccccg | tctcctcgct | gcccacctcc | tcttgcctgt |
| gccctgcaag | ccttctccct | cggccagcga | gaagatagcc | ttgaggtctc | ctctttctgc |
| tcagaagcac | tggtgacctg | tgctgctctg | acccaccccc | gggttcctcc | cctgcagccc |
| atgggcccca | cctgccccac | acctgctcca | gttccccctc | ctgaggcccc | atcgcccttc |
| agggccccac | cgttccatcc | tccgggcccc | atgccctcag | tgggctccat | gccctcagca |
| ggccccatgc | cctcagcagg | ccccatgccc | tcagcaggcc | ctgtgccctc | ggcacgccct |
| ggacctccca | ccacagccaa | ccacctaggc | ctttctgtcc | caggcctagt | gtctgtccct |
| ccccggcttc | ttcctggccc | tgagaaccac | cgggcaggct | caaatgagga | ccccatcctt |
| gcccctagtg | ggactccccc | acctactata | cccccagatg | aaacttttgg | ggggagagtg |
| cccagaccag | cctttgtcca | ctatgacaag | gaggaggcat | ctgatgtgga | gatctccttg |
| gaaagtgact | ctgatgacag | cgtggtgatc | gtgcccgagg | ggcttccccc | cctgccaccc |
| ccaccaccct | caggtgccac | accaccccct | atagccccca | ctgggccacc | aacagcctcc |
| cctcctgtgc | cagcgaagga | ggagcctgaa | gaacttcctg | cagccccagg | gcctctcccg |
| ccacccccac | ctccgccgcc | gcctgttcct | ggtcctgtga | cgctccctcc | accccagttg |
| gtccctgaag | ggactcctgg | tgggggagga | cccccagccc | tggaagagga | tttgacagtt |
| attaatatca | acagcagtga | tgaagaggag | gaggaagagg | aagaagggga | agaagaagaa |
| gaggaagaag | aggaagagga | ggaagacttt | gaggaagagg | aagaggatga | agaggaatat |
| tttgaagagg | aagaagagga | ggaagaagag | tttgaggaag | aatttgagga | agaagaaggt |
| gagttagagg | aagaagaaga | agaggaggat | gaggaggagg | aagaagaact | ggaagaggtg |
| gaagacctgg | agtttggcac | agcaggaggg | gaggtagaag | aaggtgcacc | tccaccccca |
| accctgcctc | cagctctgcc | tccccctgag | tctcccccaa | aggtgcagcc | agaacccgaa |
| cccgaacccg | ggctgctttt | ggaagtggag | gagccaggga | cggaggagga | gcgtggggct |
| gacacagctc | ccaccctggc | ccctgaagcg | ctcccctccc | agggagaggt | ggagagggaa |
| ggggaaagcc | ctgcggcagg | gccccctccc | caggagcttg | ttgaagaaga | gccctctgct |
| cccccaaccc | tgttggaaga | ggagcctgag | gatgggagtg | acaaggtgca | gcccccacca |
| gagacacctg | cagaagaaga | gatggagaca | gagacagagg | ccgaagctct | ccaggaaaag |
| gagcaggatg | acacagctgc | catgctggcc | gacttcatcg | attgtccccc | tgatgatgag |
| aagccaccac | ctcccacaga | gcctgactcc | tag | | |
| SEQ ID NO:3 (PELP1 C-terminal Amino Acid Sequence) | | | | | |
| LTVININSSD | EEEEEEEEGE | EEEEEEEEEE | EDFEEEEEDE | EEYFEEEEEE | EEEFEEEFEE |
| EEGELEEEEE | EEDEEEEEEL | EEVEDLEFGT | AGGEVEEGAP | PPPTLPPALP | PPESPPKVQP |
| EPEPEPGLLL | EVEEPGTEEE | RGADTAPTLA | PEALPSQGEV | EREGESPAAG | PPPQELVEEE |
| PSAPPTLLEE | EPEDGSDKVQ | PPPETPAEEE | METETEAEAL | QEKEQDDTAA | MLADFIDCPP |
| DDEKPPPPTE | PDS | | | | |
| SEQ ID NO:4 (PELP1 C-terminal nucleic acid) | | | | | |
| ttgacagtta | ttaatatcaa | cagcagtgat | gaagaggagg | aggaagagga | agaaggggaa |
| gaagaagaag | aggaagaaga | ggaagaggag | gaagactttg | aggaagagga | agaggatgaa |
| gaggaatatt | ttgaagagga | agaagaggag | gaagaagagt | ttgaggaaga | atttgaggaa |
| gaagaaggtg | agttagagga | agaagaagaa | gaggaggatg | aggaggagga | agaagaactg |
| gaagaggtgg | aagacctgga | gtttggcaca | gcaggagggg | aggtagaaga | aggtgcacct |
| ccacccccaa | ccctgcctcc | agctctgcct | ccccctgagt | ctcccccaaa | ggtgcagcca |
| gaacccgaac | ccgaacccgg | gctgcttttg | gaagtggagg | agccagggac | ggaggaggag |
| cgtggggctg | acacagctcc | caccctggcc | cctgaagcgc | tcccctccca | gggagaggtg |
| gagagggaag | gggaaagccc | tgcggcaggg | ccccctcccc | aggagcttgt | tgaagaagag |
| ccctctgctc | ccccaaccct | gttggaagag | gagcctgagg | atgggagtga | caaggtgcag |
| cccccaccag | agacacctgc | agaagaagag | atggagacag | agacagaggc | cgaagctctc |
| caggaaaagg | agcaggatga | cacagctgcc | atgctggccg | acttcatcga | ttgtccccct |
| gatgatgaga | agccaccacc | tcccacagag | cctgactcct | ag | |
| SEQ ID NO:5 (PELP1 N-terminal 330 amino acids) | | | | | |
| MAAAVLSGPS | AGSAAGVPGG | TGGLSAVSSG | PRLRLLLLES | VSGLLQPRTG | SAVAPVHPPN |
| RSAPHLPGLM | CLLRLHGSVG | GAQNLSALGA | LVSLSNARLS | SIKTRFEGLC | LLSLLVGESP |
| TELFQQHCVS | WLRSIQQVLQ | TQDPPATMEL | AVAVLRDLLR | YAAQLPALFR | DISMNHLPGL |
| LTSLLGLRPE | CEQSALEGMK | ACMTYFPRAC | GSLKGKLASF | FLSRVDALSP | QLQQLACECY |
| SRLPSLGAGF | SQGLKHTESW | EQELHSLLAS | LHTLLGALYE | GAETAPVQNE | GPGVEMLLSS |
| EDGDAHVLLQ | LRQRFSGLAR | CLGLMLSSEF | GAPVSVPVQE | ILDFICRTLS | VSSKNISLHG |
| DGPLRLLLLP | SIHLEALDLL | SALILACGSR | LLRFGILIGR | LLPQVLNSWS | IGRDSLSPGQ |
| ERPYSTVRTK | VYAILELWVQ | VCGAS | | | |
| SEQ ID NO:6 (PELP1 N-terminal nucleic acid | | | | | |
| sequence coding for 330 N-terminal amino acids) | | | | | |
| atggcggcag | ccgttctgag | tgggccctct | gcgggctccg | cggctggggt | tcctggcggg |
| accgggggtc | tctcggcagt | gagctcgggc | ccgcggctcc | gcctgctgct | gctggagagt |
| gtttctggtt | tgctgcaacc | tcgaacgggg | tctgccgttg | ctccggtgca | tcccccaaac |
| cgctcggccc | cacatttgcc | cgggctcatg | tgcctattgc | ggctgcatgg | gtcggtgggc |
| ggggcccaga | acctttcagc | tcttggggca | ttggtgagtc | tcagtaatgc | acgtctcagt |
| tccatcaaaa | ctcggtttga | gggcctgtgt | ctgctgtccc | tgctggtagg | ggagagcccc |
| acagagctat | tccagcagca | ctgtgtgtct | tggcttcgga | gcattcagca | ggtgttacag |
| acccaggacc | cgcctgccac | aatggagctg | gccgtggctg | tcctgaggga | cctcctccga |
| tatgcagccc | agctgcctgc | actgttccgg | gacatctcca | tgaaccacct | ccctggcctt |
| ctcacctccc | tgctgggcct | caggccagag | tgtgagcagt | cagcattgga | aggaatgaag |
| gcttgtatga | cctatttccc | tcgggcttgt | ggttctctca | aaggcaagct | ggcctcattt |
| tttctgtcta | gggtggatgc | cttgagccct | cagctccaac | agttggcctg | tgagtgttat |
| tcccggctgc | cctctttagg | ggctggcttt | tcccaaggcc | tgaagcacac | cgagagctgg |
| gagcaggagc | tacacagtct | gctggcctca | ctgcacaccc | tgctgggggc | cctgtacgag |
| ggagcagaga | ctgctcctgt | gcagaatgaa | ggccctgggg | tggagatgct | gctgtcctca |
| gaagatggtg | atgcccatgt | ccttctccag | cttcggcaga | ggttttcggg | actggcccgc |
| tgcctagggc | tcatgctcag | ctctgagttt | | | |
| SEQ ID NO:7 (PELP1-ER-blocking peptide 1) | | | | | |
| SSGPRLRLLL | LESVS | | | | |
| SEQ ID NO:8 (PELP1-ER-blocking peptide 2) | | | | | |
| PHLPGLMCLL | RLHGS | | | | |
| SEQ ID NO:9 (PELP1-ER-blocking peptide 3) | | | | | |
| FEGLCLLSLL | VGESP | | | | |
| SEQ ID NO:10 (PELP1-ER-blocking peptide 4) | | | | | |
| LAVAVLRDLL | RYAAQ | | | | |
| SEQ ID NO:11 (PELP1-ER-blocking peptide 5) | | | | | |
| ISMNHLPGLL | TSLLG | | | | |
| SEQ ID NO:12 (PELP1-ER-blocking peptide 6) | | | | | |
| SWEQELHSLL | ASLHTLLGAL | YE | | | |
| SEQ ID NO:13 (PELP1-ER-blocking peptide 7) | | | | | |
| HGDGPLRLLL | LPSIHLE | | | | |
| SEQ ID NO:14 (PELP1-H1 Mutant Polypeptide) | | | | | |
| MAAAVLSGPS | AGSAAGVPGG | TGGLSAVSSG | PRLRLLLLES | VSGLLQPRTG | SAVAPVHPPN |
| RSAPHLPGLM | CLLRLHGSVG | GAQNLSALGA | LVSLSNARLS | SIKTRFEGLC | LLSLLVGESP |
| TELFQQHCVS | WLRSIQQVLQ | TQDPPATMEL | AVAVLRDLLR | YAAQLPALFR | DISMNHLPGL |
| LTSLLGLRPE | CEQSALEGMK | ACMTYFPRAC | GSLKGKLASF | FLSRVDALSP | QLQQLACECY |
| SRLPSLGAGF | SQGLKHTESW | EQELHSLLAS | LHTLLGALYE | GAETAPVQNE | GPGVEMLLSS |
| EDGDAHVLLQ | LRQRFSGLAR | CLGLMLSSEF | GAPVSVPVQE | ILDFICRTLS | VSSKNISLHG |
| DGPLRLLLLP | SIHLEALDLL | SALILACGSR | LLRFGILIGR | LLPQVLNSWS | IGRDSLSPGQ |
| ERPYSTVRTK | VYAILELWVQ | VCGASAGMLQ | GGASGEALLT | HLLSDISPPA | DALKLRSPRG |
| SPDGSLQTGK | PSAPKKLKLD | VGEAMAPPSH | RKGDSNANSD | VCAAALRGLS | RTILMCGPLI |
| KEETHRRLHD | LVLPLVMGVQ | QGEVLGSSPY | TSSRCRRELY | CLLLALLLAP | SPRCPPPLAC |
| ALQAFSLGQR | EDSLEVSSFC | SEALVTCAAL | THPRVPPLQP | MGPTCPTPAP | VPPPEAPSPF |
| RAPPFHPPGP | MPSVGSMPSA | GPMPSAGPMP | SAGPVPSARP | GPPTTANHLG | LSVPGLVSVP |
| PRLLPGPENH | RAGSNEDPIL | APSGTPPPTI | PPDETFGGRV | PRPAFVHYDK | EEASDVEISL |
| ESDSDDSVVI | VPEGLPPLPP | PPPSGATPPP | IAPTGPPTAS | PPVPAKEEPE | ELPAAPGPLP |
| PPPPPPPPVP | GPVTLPPPQL | VPEGTPGGGG | PPALEEDLTV | | |
| SEQ ID NO:15 (PELP1-H1 Mutant Nucleic Acid Sequence) | | | | | |
| atggcggcag | ccgttctgag | tgggccctct | gcgggctccg | cggctggggt | tcctggcggg |
| accgggggtc | tctcggcagt | gagctcgggc | ccgcggctcc | gcctgctgct | gctggagagt |
| gtttctggtt | tgctgcaacc | tcgaacgggg | tctgccgttg | ctccggtgca | tcccccaaac |
| cgctcggccc | cacatttgcc | cgggctcatg | tgcctattgc | ggctgcatgg | gtcggtgggc |
| ggggcccaga | acctttcagc | tcttggggca | ttggtgagtc | tcagtaatgc | acgtctcagt |
| tccatcaaaa | ctcggtttga | gggcctgtgt | ctgctgtccc | tgctggtagg | ggagagcccc |
| acagagctat | tccagcagca | ctgtgtgtct | tggcttcgga | gcattcagca | ggtgttacag |
| acccaggacc | cgcctgccac | aatggagctg | gccgtggctg | tcctgaggga | cctcctccga |
| tatgcagccc | agctgcctgc | actgttccgg | gacatctcca | tgaaccacct | ccctggcctt |
| ctcacctccc | tgctgggcct | caggccagag | tgtgagcagt | cagcattgga | aggaatgaag |
| gcttgtatga | cctatttccc | tcgggcttgt | ggttctctca | aaggcaagct | ggcctcattt |
| tttctgtcta | gggtggatgc | cttgagccct | cagctccaac | agttggcctg | tgagtgttat |
| tcccggctgc | cctctttagg | ggctggcttt | tcccaaggcc | tgaagcacac | cgagagctgg |
| gagcaggagc | tacacagtct | gctggcctca | ctgcacaccc | tgctgggggc | cctgtacgag |
| ggagcagaga | ctgctcctgt | gcagaatgaa | ggccctgggg | tggagatgct | gctgtcctca |
| gaagatggtg | atgcccatgt | ccttctccag | cttcggcaga | ggttttcggg | actggcccgc |
| tgcctagggc | tcatgctcag | ctctgagttt | ggagctcccg | tgtccgtccc | tgtgcaggaa |
| atcctggatt | tcatctgccg | gaccctcagc | gtcagtagca | agaatattag | cttgcatgga |
| gatggtcccc | tgcggctgct | gctgctgccc | tctatccacc | ttgaggcctt | ggacctgctg |
| tctgcactca | tcctcgcgtg | tggaagccgg | ctcttgcgct | ttgggatcct | gatcggccgc |
| ctgcttcccc | aggtcctcaa | ttcctggagc | atcggtagag | attccctctc | tccaggccag |
| gagaggcctt | acagcacggt | tcggaccaag | gtgtatgcga | tattagagct | gtgggtgcag |
| gtttgtgggg | cctcggcggg | aatgcttcag | ggaggagcct | ctggagaggc | cctgctcacc |
| cacctgctca | gcgacatctc | cccgccagct | gatgccctta | agctgcgtag | cccgcggggg |
| agccctgatg | ggagtttgca | gactgggaag | cctagcgccc | ccaagaagct | aaagctggat |
| gtgggggaag | ctatggcccc | gccaagccac | cggaaagggg | atagcaatgc | caacagcgac |
| gtgtgtgcgg | ctgcactcag | aggcctcagc | cggaccatcc | tcatgtgtgg | gcctctcatc |
| aaggaggaga | ctcacaggag | actgcatgac | ctggtcctcc | ccctggtcat | gggtgtacag |
| cagggtgagg | tcctaggcag | ctccccgtac | acgagctccc | gctgccgccg | tgaactctac |
| tgcctgctgc | tggcgctgct | gctggccccg | tctcctcgct | gcccacctcc | tcttgcctgt |
| gccctgcaag | ccttctccct | cggccagcga | gaagatagcc | ttgaggtctc | ctctttctgc |
| tcagaagcac | tggtgacctg | tgctgctctg | acccaccccc | gggttcctcc | cctgcagccc |
| atgggcccca | cctgccccac | acctgctcca | gttccccctc | ctgaggcccc | atcgcccttc |
| agggccccac | cgttccatcc | tccgggcccc | atgccctcag | tgggctccat | gccctcagca |
| ggccccatgc | cctcagcagg | ccccatgccc | tcagcaggcc | ctgtgccctc | ggcacgccct |
| ggacctccca | ccacagccaa | ccacctaggc | ctttctgtcc | caggcctagt | gtctgtccct |
| ccccggcttc | ttcctggccc | tgagaaccac | cgggcaggct | caaatgagga | ccccatcctt |
| gcccctagtg | ggactccccc | acctactata | cccccagatg | aaacttttgg | ggggagagtg |
| cccagaccag | cctttgtcca | ctatgacaag | gaggaggcat | ctgatgtgga | gatctccttg |
| gaaagtgact | ctgatgacag | cgtggtgatc | gtgcccgagg | ggcttccccc | cctgccaccc |
| ccaccaccct | caggtgccac | accaccccct | atagccccca | ctgggccacc | aacagcctcc |
| cctcctgtgc | cagcgaagga | ggagcctgaa | gaacttcctg | cagccccagg | gcctctcccg |
| ccacccccac | ctccgccgcc | gcctgttcct | ggtcctgtga | cgctccctcc | accccagttg |
| gtccctgaag | ggactcctgg | tgggggagga | cccccagccc | tggaagagga | tttgac |

| SEQ ID NO:16 (PELP1 siRNA1) | |
|---|---|
| r (GGAGGAGCCU GAAGAACUU) dTT; r (AAGUUCUUCA | |
| GGCUCCUCC) dTT | |
| SEQ ID NO:17 (PELP1 siRNA2) | |
| r (UUCCUGGAGC AUCGGUAGA) dTT; r (UCUACCGAUG | |
| CUCCAGGAA) dTT | |
| SEQ ID NO:18 (PELP1 siRNA3) | |
| r (GGCAAGCUGG CCUCAUUUU) dTT; r (AAAAUGAGGC | |
| CAGCUUGCC) dTT | |
| SEQ ID NO:19 (PELP1 siRNA4) | |
| r (GGAAUGAAGG CUUGUAUGA) dTT; r (UCAUACAAG | |
| CCUUCAUUCC) dTT | |
| SEQ ID NO:20 (PELP1 antibody generating epitope) | |
| RDSLSPGQER | PYSTVRTKV |

1. An isolated polypeptide comprising the amino acid sequence of SEQ ID NOS: 3, 5, 7-13, or 14.

2. A PELP1-specific antibody capable of binding a PELP1 polypeptide of at least 7 amino acids of SEQ ID NOS: 1, 3, 5, 7-13, 14, or 20.

3. The antibody of claim 2 wherein the antibody is monoclonal.

4. An isolated nucleic acid comprising the nucleotide sequence of SEQ ID NOS: 4, 6, or 15.

5. A siRNA capable of disrupting PELP1 activity.

6. The siRNA of claim 5 comprising an siRNA of SEQ ID NOS: 16, 17, 18, or 19.

7. A peptidomimetic capable of disrupting PELP1 activity.

8. A pharmaceutical composition comprising an isolated polypeptide of claim 1 and a pharmaceutically acceptable carrier.

9. A pharmaceutical composition comprising a PELP1-specific antibody of claim 2 and a pharmaceutically acceptable carrier.

10. A pharmaceutical composition comprising a siRNA of claim 5 and a pharmaceutically acceptable carrier.

11. A pharmaceutical composition comprising a peptidomimetic of claim 7 and a pharmaceutically acceptable carrier.
